(19) United States
(12) Patent Application Publication
(10) Pub. No.: US 2008/0057047 A1
(43) Pub. Date: Mar. 6, 2008

(54) Use of bacillus amyloliquefaciens PB6 for the prophylaxis or treatment of gastrointestinal and immuno-related diseases

(76) Inventors: Benedikt Sas, Stekene (BE); Johan Van Hemel, Antwerpen (BE); Jan Vandenkerckhove, Testelt (BE); Eric Peys, Balen (BE); Hai Meng Tan, Singapore (SG); Chea-Yun Se, Johor (MY); Chanivilparampu Nanappan Ramchand, Chennai (IN); Jerry Varghese, Kerala (IN)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/605,736

(22) Filed: Nov. 28, 2006

Related U.S. Application Data
(60) Provisional application No. 60/740,518, filed on Nov. 29, 2005.

(51) Int. Cl. A61K 35/00; A61K 31/35; A61P 1/00; C12N 1/20

(52) U.S. Cl. 424/93.462; 424/93.46; 435/252.5; 514/460

(57) ABSTRACT

Bacteria of the sp. Bacillus that produce a lipopeptide are found to be effective in the treatment and prophylaxis of gastro-intestinal disease when administered as a probiotic. In particular, a strain of Bacillus bacteria identified as PB6 is useful for the treatment of Antibiotic Associated Diarrhea (AAD) or the more serious condition Clostridium difficile associated diarrhea (CDAD) when administered as a probiotic. Additionally, these bacteria have been found efficient for the treatment of immunorelated diseases such as Inflammatory Bowel Disease (IBD).

### BACKGROUND OF THE INVENTION

The invention relates generally to the administration of bacteria to treat gastrointestinal disease and, more specifically, to the administration of bacteria of a strain of Bacillus amyloliquefaciens to treat antibiotic associated diarrhea (AAD) and Clostridium difficile associated disease (CDAD).

The term antibiotic-associated diarrhea refers to a benign, self-limited diarrhea, following the use of antimicrobials. Typically no pathogens are identified and the diarrhea is due to changes in the composition and function of the intestinal flora. Most patients respond to supportive measures and discontinuation of antibiotics.

The prolonged use of multiple antibiotics, especially broad-spectrum agents with poor intestinal absorption or high biliary excretion, induces a change in the composition and function of the intestinal flora and therefore results in a higher incidence of AAD.^{1,2} The degree of alteration will be influenced by the ability of the normal flora to resist colonization and the type of antibiotic used. A decrease in the colonic anaerobic flora interferes with carbohydrate and bile acid metabolism. Osmotic or secretory diarrhea may occur. Overgrowth of opportunistic pathogens takes place as a result of microbiologic and metabolic alterations.

C difficile, an anaerobic gram-positive rod, accounts for 15% to 20% of all AAD cases. In particular this organism can be isolated in a great number of AAD cases with evidence of colitis and in all those with pseudomembranes. It is widely present in the environment, may survive for a considerable time, and is transmitted by the fecal-oral route to susceptible individuals. It is considered part of the normal flora of infants and can be isolated in about 5% of healthy adults and in up to one third of asymptomatic or colonized, hospitalized patients.

The clinical manifestations of AAD may vary from mild diarrhea to fulminant colitis.³ The severity of C difficile colitis appears to be influenced by a myriad of factors including age, comorbidity, host's immune response, and the use of antiperistaltic agents. Interestingly, bacterial genotype and toxin production appear to play minimal roles.⁴ The cardinal symptom of the disease is diarrhea that commonly develops during treatment but may appear as late as 8 weeks after discontinuation of antibiotics. In most cases of AAD, patients present with loose stools, minimal signs of colitis, and no constitutional symptoms. The diarrhea promptly responds to supportive measures and withdrawal of the antimicrobial agent.

Clostridium difficile was first described in 1935⁵, but it was not associated with antibiotic-related diarrhea until the late 1970's. Clostridium difficile is a spore-forming gram-positive anaerobic Bacillus that produces at least two exotoxins: toxin A, primarily an enterotoxin, and toxin B, a cytotoxin. The organism causes gastro-intestinal infections in humans that range in severity from asymptomatic colonization to severe diarrhea, pseudomembranous colitis (PMC), toxic megacolon, colonic perforation, and death^{6,7,8}. The first step in development of C. difficile colonization is disruption of the normal flora of the colon, usually caused by antibiotics or, in unusual cases, by antineoplastic or immunosuppressive drugs^{9,10}. Colonization occurs by the fecal-oral route; ingested spores of C. difficile survive the gastric acid barrier and germinate in the colon^{11,12}. Symptoms of CDAD may start on the first day of antibiotic therapy or up to several weeks after antibiotic therapy is stopped¹³. The following two factors recently have been shown to increase the probability of symptomatic disease in patients who acquire C. difficile colonization in hospital; the severity of other illnesses, and reduced levels of serum IgG antibody to toxin A¹⁴. These results suggest that pre-existing anti-toxin A antibody may ameliorate severity of disease and that immunization might be efficacious in preventing and controlling nosocomial CDAD.

For clarity, we define patients as having C. difficile-associated disease (CDAD) if they display symptomatic illness caused by C. difficile. Detection of the presence of a C. difficile toxin in the stool of patients with diarrhea has been the most generally accepted method of diagnosis.

Clostridium difficile is the cause of approximately 25% of all cases of antibiotic-associated diarrhea¹⁵. Most cases of C. difficile-associated disease occur in hospitals or long-term care facilities (rate of 25-60 per 100,000 occupied bed-days), causing more than 300,000 cases per year in the US and similar rates estimated for many European countries. It can add up to two weeks to the length of the hospitalization, at an additional cost of $6,000-$10,000 per case^{16,17,18,19,20,21}

Diarrhea may resolve spontaneously in patients with CDAD once the inciting antibiotic has been withdrawn, and for some patients with mild disease no specific therapy may be necessary^{22,23}. However, the standard practice is to treat almost all symptomatic patients with the antibiotics vancomycin or metronidazole. Although metronidazole is not currently approved by the FDA for the treatment of CDAD, it is widely used as first-line therapy due to the higher cost of vancomycin and concerns over the emergence of vancomycin resistant bacteria. Because metronidazole effectively disrupts normal enteric flora, it also predisposes patients to colonization with metronidazole resistant enterococci²⁴. Oral metronidazole (250 mg 4 times per day or 500 mg 3 times per day) for 10-14 d is usually adequate. Oral vancomycin hydrochloride (125 mg 4 times per day) for 10-14 d is indicated for those who cannot tolerate oral metronidazole, those in whom metronidazole therapy fails, pregnant patients, and, perhaps, severely ill patients. The first relapse/recurrence of Clostridium difficile colitis can be treated with another 10- to 14-d course of oral metronidazole or vancomycin.

Treatment of CDAD with vancomycin or metronidazole propagates a vicious cycle by altering normal, protective flora of the gut. Consequently 20% of treated patients with an initial episode have a recurrence of CDAD, usually within two weeks after discontinuation of therapy^{25,26,27,28}. A further benefit of removing antibiotics from the treatment regimen of CDAD is a reduction in selective pressure for bacterial resistance. Vancomycin and metronidazole have been clearly shown to select for resistant gram-positive cocci, such as VRE. Retrospective epidemiological studies have linked intestinal VRE colonization with the use of broad-spectrum antibiotics such as the cephalosporins, fluoroquinolones and metronidazole²⁹. Intestinal VRE colonization provides a reservoir for this pathogen within the hospital. Many strains of VRE are multiresistant, leaving few options for treatment of life-threatening systemic infections. C. difficile patients, perhaps because of their prior antibiotic disposure, appear to be especially susceptible to VRE colonization and infection^{30,31}. Management of VRE colonization is a critical component of hospital infection control practices. Therefore, therapeutic strategies that reduce the risk of VRE colonization both in the general patient population and in C. difficile patients are highly desirable. The potential emergence of vancomycin and metronidazole resistant C. difficile presents an additional risk for the use of antibiotics to treat this disease. Currently, the occurrence of antibiotic resistant C. difficile is sporadic but has been reported in up to 12% of clinical isolates³².

### SUMMARY OF THE INVENTION

The invention relates to the prophylaxis of a bowel condition, such as antibiotic associated diarrhea, Clostridium difficile acquired diarrhea, inflammatory bowel disease, and gastro-intestinal disease, by administering an effective amount of a Bacillus bacteria that produces lipopeptides. The Bacillus bacteria may be administered as a probiotic and may be combined with other probiotics, such as inulin.

Using biochemical methods, and specifically API 50 CBH/L, a preferred Bacillus was putatively identified as Bacillus subtilis. Using 16S rRNA, the preferred Bacillus was also putatively identified as Bacillus subtilis. Using gyrA, the preferred Bacillus was putatively identified as Bacillus amyloliquefaciens. Prior to the gyrA assay, the preferred Bacillus was deposited with the ATCC and identified as Bacillus subtilis.

The invention also relates to an isolated bacteria strain having the 16S rRNA sequence of SEQ ID NO. 1, to an isolated bacterial strain having 90% homology, 80% homology, 70% homology, 60% homology and 50% homology to SEQ ID NO. 1.

The invention also relates to an isolated bacteria strain having the partial gyrA sequence of SEQ ID NO. 2, to an isolated bacterial strain having 90% homology, 80% homology, 70% homology, 60% homology and 50% homology to SEQ ID NO. 2.

The invention also relates to an isolated bacteria strain having the partial gyrA sequence of SEQ ID NO. 3, to an isolated bacterial strain having 90% homology, 80% homology, 70% homology, 60% homology and 50% homology to SEQ ID NO. 3.

The invention further relates to a Bacillus bacteria of the strain identified as ATCC strain PTA-6737.

The preferred Bacillus PB6 can be positioned for several unmet medical needs due to its versatile and unique characteristics.

### BRIEF DESCRIPTION OF THE FIGURES

FIG. 1 is a photograph of the antagonistic effect of Bacillus PB6 (1) against C. perfringens ATCC13124 (2) and C. difficile ATCC9689 (3).

FIG. 2 is a photograph of the antagonistic effect of Bacillus PB6 on Clostridium difficile NAP1/027.

FIG. 3 is a photograph of the antagonistic effect of Bacillus PB6 on Campylobacter jejuni ATCC55918.

FIG. 4 is a drawing of the chemical structure of surfactin.

FIG. 5 is a chart of the % survival in hamsters suffering from CDAD with different treatments.

FIG. 6 is the 1466-bp 16S rRNA gene sequence (nucleotide position of 27-1492) of Bacillus PB6.

FIG. 7 is the 1023-bp partial gyrA sequence (nucleotide position of 43-1065) of Bacillus PB6.

FIG. 8 is the 801-bp consensus sequence obtained from partial gyrA sequencing of Bacillus PB6.

FIG. 9 is a gel of the detection of a PCR product (1650-bp) encoding for hemolysin BL; Lane 1, the GeneRuler™ mass ladders (3000, 2000, 1500, 1200-bp); Lane 2, Bacillus PB6; Lane 3, Escherichia coli ATCC 25922; Lane 4, B. cereus ATCC 49064; Lane 5, B. cereus ATCC 11778. No amplified band corresponding to a 1650-bp PCR product was detected in any of the lanes except for lanes 4 and 5.

FIG. 10 is a gel of the detection of a PCR product (1437-bp) encoding for non-hemolytic enterotoxin (Nhe). Lane 1, the GeneRuler™ mass ladders (3000, 2000, 1500, 1200-bp); Lane 2, Bacillus PB6; Lane 3, Escherichia coli ATCC 25922; Lane 4, B. cereus ATCC 49064, Lane 5, B. cereus ATCC 11778. No amplified band corresponding to a 1437-bp PCR product was detected in any of the lanes.

FIG. 11 is a gel of the detection of a PCR product (1400-bp) encoding for enterotoxin K (EntK); Lane 1, Bacillus PB6; Lane 3, Escherichia coli ATCC 25922; Lane 4, B. cereus ATCC 49064; Lane 5, B. cereus ATCC 11778; Lane 6, GeneRuler™ mass ladders (3000, 2000, 1500, 1200-bp); no amplified band corresponding to a 1400-bp PCR product was detected in any of the lanes.

FIG. 12 is a photograph showing the lack of antagonistic effect of Bacillus cereus (1) against C. perfringens ATCC13124 (2) and C. difficile ATCC9689 (3).

FIG. 13 is a photograph of the antagonistic effect of Bacillus PB6 against Campylobacter jejuni ATCC 33291.

FIG. 14 is a photograph showing the lack of antagonistic effect of Bacillus cereus against Campylobacter jejuni ATCC 33291.

### DETAILED DESCRIPTION OF PREFERRED EMBODIMENTS

PB6 is a proprietary bacterial strain that was isolated from nature and has not been genetically modified. Using a ribotyping technique this bacteria was identified as being a strain of Bacillus subtilis. DNA:DNA hybridization studies indicate that Bacillus PB6 strain may more likely be a Bacillus amyloliquefaciens, which will be further described below.

As used in this specification, the term prophylaxis means a medical or public health procedure whose purpose is to prevent rather than treat or cure a condition. As used in this specification, the term treatment means a medical or public health procedure whose purpose is to treat or cure a condition. As used in this specification, the term synergistic compound means a compound which enhances the prophylactic effect or treatment efficacy of a Bacillus bacterium administered for the prophylaxis or treatment of a disease or health condition. As used in this specification, lipopeptides are molecules that contain both lipids and proteins and include surface-active molecules containing several amino acids and one or more fatty acids. Surfactins, iturins, mycosubtilins, baillomycins, bacillopeptins, fengycins, and plipastatins are examples of lipopeptides.

### Example 1

### Efficacy of Bacillus Amyloliquefaciens PB6 Against C. Difficile, AAD and CDAD

The antagonistic properties of Bacillus PB6 were tested against C. perfringens ATCC13124 and C. difficile ATCC9689.

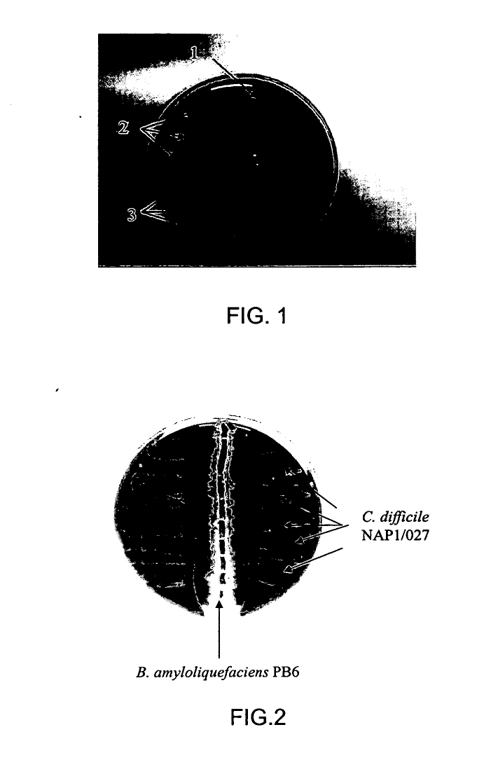

Bacillus PB6 had antagonistic effect against C. perfringens ATCC 13124 and C. difficile ATCC9689. A clear zone was observed at the intersections of the streak-lines on the plate for both species. An example of the test plate is depicted in FIG. 1.

Bacillus PB6 also had antagonistic effect against C. difficile NAP 1/027. This C. difficile strain is linked to several highly dangerous outbreaks and shows resistance to antibiotics. An example of the test plate is depicted in FIG. 2.

In order to determine the antimicrobial effect of the secondary metabolites of Bacillus PB6, the bacteria was fermented and the fermentation product was extracted by diethyl ether. The organic layer was separated, concentrated in vacuo and redissolved in DMSO for screening.

The minimum inhibitory concentration (MIC) of the extract was 2.5-5 μg/ml against C. perfringens and 5-10 μg/ml against C. difficile (Table 1).

TABLE 1
Results of the screening with the crude extract of Bacillus PB6
against C. perfringens, C. difficile and C. jejuni

| | MIC (μg/ml) | | |
|---|---|---|---|
| | C. perfringens | C. difficile | C. jejuni |
| | ATCC13124 | ATCC9689 | ATCC 33291 |
| Crude extract | 2.5-5 | 5-10 | 25-100 |

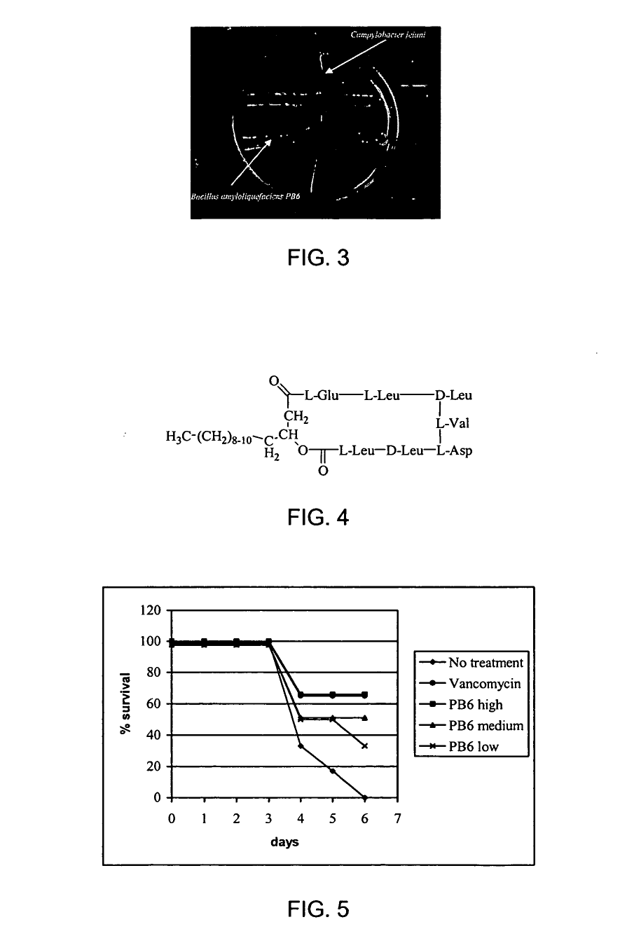

It was also proven that Bacillus PB6 inhibits the growth of Campylobacter jejuni in vitro. An example of the test plate is shown in FIG. 3. The MIC of an ether extract of the fermentation product of Bacillus PB6 against C. jejuni was 25-100 μg/ml.

Campylobacter jejuni and Helicobacter pylori are very closely related and therefore it is probable that Bacillus PB6 is also active against Helicobacter pylori. Furthermore, in literature can be found that Bacillus bacteria (e.g. Bacillus subtilis) possess activity against Helicobacter pylori.³³

Further research of the crude extract showed that the molecule responsible for the activity against Clostridium was the cyclic lipopeptide surfactin (FIG. 4).

When the activity of pure surfactin (either purified from our fermentation or purchased from Sigma) was determined against Clostridium, a higher MIC was found. The MIC against C. perfringens proved to be 10-25 μg/ml. It is remarkable that the pure active is less active than the crude fermentation extract. This is likely due to a co-factor(s) present in the extract that enhances the activity of surfactin. This has been shown by experiments where the MIC of surfactin was compared with the MIC of surfactin in combination with an inactive compound. This inactive compound was isolated from the same extract we isolated surfactin from. The MIC of this combination was between 1 and 10 μg/ml.

Experiments have shown that, when the filtrates of Bacillus PB6 were treated with pepsin and trypsin, the activity against Clostridium sp. decreased significantly. Since pepsin and trypsin are enzymes produced in the mucosal lining of the stomach and the pancreas, it is clear that oral administration of surfactin will lead to a significant loss of the activity. Other experiments, where surfactin has been incubated at 37° C. for 30, 60 and 90 minutes with 0.1 N HCl, showed that acidic conditions (as in the stomach) lead to a loss in activity (Table 2). Therefore, administration of Bacillus PB6 (eventually as spores) is more effective to obtain effective concentrations of surfactin or other lipopeptides in the intestine.

TABLE 2
Results of the influence of acidic incubation on the antibacterial activity of
surfactin against Clostridium perfringens

MIC (μg/ml) (C. perfringens)

| | No HCL | 30′ HCl | 60′ HCl | 90′ HCl |
|---|---|---|---|---|
| surfactin | 1-10 | 10-25 | 25-50 | 25-50 |

Besides the production of antimicrobial secondary metabolites, Bacillus PB6 spores are able to germinate in the intestine and thus can suppress pathogens by competitive exclusion.

Bacillus, more specifically Bacillus subtilis, remains one of the most potent and beneficial of all health-promoting and immune-stimulating bacteria. According to several clinical studies documented in medical research reports, the cell wall components of ingested Bacillus are able to activate nearly all systems of the human immune defense, including the activation of at least three specific antibodies (IgM, IgG and IgA) which are highly effective against many of the harmful viruses, fungi and bacterial pathogens which regularly attempt to invade and infect the human system. Bacillus subtilis has also been shown to stimulate B and T lymphocytes and macrophages. Also evidence has been provided that Bacillus subtilis spores may exert an immunomodulatory effect in vivo. And an increased response of plaque-forming cells to T dependent antigens has been described after exposure to spores as well as an enhancement of different phagocytes' functions.^{34,35,36,37,38,39,40,41}

In a study (Table 3) an elevated degree in phagocytosis was observed in broilers fed with different levels of B. amyloliquefaciens PB6 compared to the antibiotic and negative controls.

From this we can conclude that also Bacillus amyloliquefaciens possesses immuno-stimulating properties.

TABLE 3
Influence of Bacillus PB6 on immune response in male broilers

| Treatment | Phagocytosis |
|---|---|
| Negative control | 6.59 |
| Positive control (100 mg/kg zinc bacitracin) | 6.23 |
| PB6 (10⁷ CFU/T) | 11.82 |
| PB6 (10⁸ CFU/T) | 8.85 |

The filtrates from Bacillus PB6 were evaluated for the presence of hemolytic, non-hemolytic enterotoxins and enterotoxin K using commercially available immunological assays (TECRA and Oxoid). The same filtrates were further subjected to cytotoxicity tests on Vero and HEp-2 cell lines. Finally, PCR-based methods were used to confirm for the presence of genes with possible enterotoxigenic capacity in Bacillus PB6. There was no immuno-cross reactivity observed with Hbl or Nhe enterotoxins and antibodies in the two commercial immunoassays. No cytotoxicity was also observed with the Vero and HEp-2 cell assays. Bacillus PB6 strain does not produce the hemolytic, non-hemolytic enterotoxins and enterotoxin K under the same conditions that allowed detection for a known toxigenic strain of B. cereus.

The in vivo toxicity of Bacillus PB6 has also been tested. Therefore a spray-dried product containing 10¹⁰ cfu/g of Bacillus PB6 was made from the fermentation product.

Toxicity Study of Spray-Dried B. Amyloliquefaciens in Wistar Rats

A first study was designed and conducted to determine the acute oral toxicity of the spray-dried Bacillus PB6 product (10¹⁰ cfu/g) in Wistar rats. A total of 5 male and 5 female animals were administered an oral dose of 5000 mg/kg (as a suspension in double distilled water and using a dose volume of 10 ml/kg). The control group consisted of 5 male and 5 female animals that received double distilled water at a dose of 10 ml/kg. No mortality was observed in the 5000 mg/kg treated animals. 50% of the animals receiving the Bacillus PB6 product were more active in comparison with the control group. None of the animals treated with Bacillus PB6 suffered from diarrhea. After necroscopy, no pathological changes were seen in any organ in both groups. Thus the maximum non-lethal dose (LD₀) and LD₅₀ of the orally administered Bacillus PB6 product in Wistar rats was found to be greater than 5000 mg/kg.

Conclusion: Maximum non-lethal dose (LD₀) and LD₅₀ of B. PB6 dry (10¹⁰ cfu/g) in Wistar rat by oral route was found to be greater than 5000 mg/kg.

Repeated Dose 28-Day Oral Toxicity of B. PB6 in Wistar Rats

This study was designed and conducted to determine repeated dose (28 days) oral toxicity of B. PB6 (10¹⁰ cfu/g) in Wistar rats. In each group 6 male and 6 female animals were administered oral doses of 250, 500 or 1000 mg/kg for 28 days. The control vehicle group also consisted of 6 male and 6 female animals, which received doubled distilled water at a dose of 10 ml/kg for 28 days. These groups were sacrificed on day 29.

The study also consisted of two reversible groups for control vehicle and high dose which each had 6 male and 6 female animals. The high dose group received medication up to day 28 and was without treatment from day 29 to 42 and sacrificed on day 43. The control vehicle group received distilled water at a dose of 10 ml/kg up to day 28 and was without treatment from day 29 to day 42 and sacrificed on day 43.

Conclusion: No Observed Adverse Event Level (NOAEL) for B. PB6 (10¹⁰ cfu/g) in 28 days toxicity trials in rats is found to be greater than 1000 mg/kg.

Local Irritancy Study (Dermal and Eye) of B. PB6 in New-Zealand White Rabbit

A total of 3 New-Zealand White Rabbits (either sex)—Same rabbits were used for dermal and eye irritation in this study. A minimum gap of 5 days was kept between both studies. 1 g of B. PB6 (10¹⁰ cfu/g) was applied as a paste to the skin for dermal irritation and 100 μl of 10% B. PB6 suspension instilled into left eye for eye irritation.

Test Item PB6 paste was applied to skin at dorso-lateral area after removal of hairs in 3 rabbits. Suspension of 10% PB6 was instilled in the eye of 3 rabbits. The test item was removed from skin of the animals 4 hours post-application. After instillation of test item in left eye, the eyelids were held together for 2-3 seconds.

The rabbits were observed and scored for dermal irritancy at 1, 24, 48 and 72 hours after removal of test item PB6. For eye irritancy scoring was carried out at the same time points post-instillation of PB6 suspension.

Conclusions: Dermal: No irritation was observed, when 1 g of B. PB6 (10¹⁰ cfu/g) was applied as a paste.

Eye: No irritation was observed, when 100 μl of 10% suspension of B. PB6 (10¹⁰ cfu/g) was instilled.

Erythrocyte Micronucleus Assay of B. Amyloliquefaciens PB6 in Mice

This study was designed and conducted to detect the damage induced by the test substance to the chromosomes or the mitotic apparatus of Swiss albino mice. A total of 5 male and 5 female animals were administered oral dose of 2500 and 5000 mg/kg, the control vehicle group consisted of 5 male and 5 female mice which received double distilled water at a dose of 10 ml/kg orally. The positive control group (5 male+5 female) received cyclophosphamide orally at a dose of 40 mg/kg.

The animals were sacrificed by excess of CO₂ at respective time points (control group, 2500 mg/kg PB6 cyclophosphamide at 24 h and 5000 mg/kg PB6 at both 24 and 48 h) and both femora were removed and bone marrow smears made on slides, stained with Giemsa and May-Greunwald stain, viewed under microscope for the incidence of micronucleus by counting 2000 immature erythrocytes.

The percentage of immature among total (immature+mature) erythrocytes is also determined for each animal by counting at least 200 erythrocytes.

Conclusion: B. PB6 (10¹⁰ cfu/g) at a dose of 2500 & 5000 mg/kg did not significantly induce micro nucleated polychromatic erythrocytes in mice.

Determination of the In Vivo Efficacy of Orally Administered Bacillus PB6 in the Treatment of Golden Syrian Hamsters Suffering from CDAD.

Study Design

Forty-two male Golden Syrian hamsters were obtained from the National Centre for Laboratory Animal Sciences, NIN (Hyderabad, India). At the beginning of the treatment period, the animals were 12 to 14 weeks old. Upon their arrival at the test facility, the animals were given a complete clinical examination under the supervision of a veterinarian to ensure that they were in good condition. The animals were acclimatized to the study conditions for a period of at least 7 days. Body weights were recorded before allocation of the animals into the study groups at the start of the trial. The animals were housed individually in polycarbonate cages (290×22×140 mm, L×W×H). The animal room and test room conditions were set as follows: temperature: 22±4° C., relative humidity: 50±20%, light/dark cycle: 12 hr/12 hr (light 07.00-19.00) and ventilation: approximately 7 cycles/hour of filtered, non-recycled air. All animals had free access to Hamster Pellet Feed (NIN, Hyderabad) and Aquaguard purified water ad libitum.

Animals were allocated to one of seven study groups (A to G). In groups A to E Clostridium difficile associated diarrhea was induced by administering 10000 CFU's of Clostridium difficile ATCC 9689 orally on day 0, followed by a subcutaneous injection in the trunk region just behind the ears of clindamycin 100 mg/Kg on day 1. Group A received no further treatment.

Group B was treated once daily from days 2 to 6 with vancomycin 50 mg/Kg by oral gavage. Groups C, D and E were treated with PB6 at a dose of 1.5×10⁸, 1.5×10⁷ and 1.5×10⁶ CFU/Kg respectively, 3 times daily with 4 hours interdose (first dose at 9.30 am) from day 1 to 6 by oral gavage. On day 1 the first dose of PB6 was given 1 hour after the injection of clindamycin. In groups F and G the animals were given PB6 at a dose of 1.5×10⁹ CFU/Kg, 3 times daily with 4 hours interdose (first dose at 9.30 am) from day 1 to 6 by oral gavage. Day 6 was the last day of treatment. Twice daily, up to day 15, observations were made for clinical signs and mortality. Signs of diarrhea were scored as being mild, moderate or severe. Body weights of animals were recorded on day 0, 7 and 14. For groups A to E faeces were tested on days 1, 2 and 7 for the presence of Clostridium toxin A and B using Immunocards (Meridian life sciences). On day 1 fecal samples were taken before clindamycin administration. On days 2 and 7 fecal samples were taken between the second and third treatment dose.

Test Preparations

A Culti-loop (Oxoid, Basingstoke, England) containing Clostridium difficile ATCC 9689 was inoculated according to the manufacturers instructions and the broth was diluted with saline to obtain 10000 CFU/ml. Clindamycin hydrochloride (Pharmacia, Puurs-Belgium) and vancomycin (Neon Laboratories, Bombay, India) were suspended in double distilled water to a concentration of 10 and 50 mg/ml respectively. Dose volumes were 10 and 1 ml/Kg bodyweight for clindamycin and vancomycin respectively. PB6 Dry (Kemin Consumer Care, Des Moines, USA), a Bacillus ‘PB6’ fermentation broth dried on a malto- and cyclodextrin carrier, was suspended in double distilled water to concentrations of 1.5E8, 1.5E6 and 1.5E5 CFU/ml. Dose volume was 10 ml/Kg bodyweight. Fresh preparations were made prior to each administration. Clindamycin, vancomycin and PB6 were administered on the basis of the last individual body weight taken. The preparations were stirred vigorously before each dosing.

Results and Discussion

About 6 hours after clindamycin was administered mild diarrhea was observed in 3 animals of the no treatment group and in 1 animal of the group to be treated with vancomycin. In groups C, D, E, where the animals received their first dose of PB6 about 1 hour after the administration of clindamycin, none of the animals showed any sign of diarrhea that same day. The number of animals suffering from diarrhea and the severity of diarrhea evolved differently between treatment groups the following 2 days (Table 4). All groups in which Clostridium difficile associated diarrhea was induced showed signs of diarrhea. The intensity of diarrhea was less in the group treated with vancomycin and the group treated with the high dose of PB6. In the no treatment group as well as in the low and mid dose PB6 groups the stool was very watery and whole of the abdominal area was wet.

TABLE 4
Number of animals with diarrhea and their score for days 1 to 3,
in the groups in which CDAD was induced.

| | Day 1 | | Day 2 | | Day 3 | |
|---|---|---|---|---|---|---|
| Treatment | number | score | number | score | number | score |
| No treatment | 3 | + | 6 | + (1) | 6 | ++ (5) |
| | | | | ++ (5) | | +++ (1) |
| Vancomycin | 1 | + | 2 | ++ | 2 | +++ |
| PB6 high | 0 | | 3 | + (2) | 4 | + (1) |
| | | | | ++ (1) | | ++ (3) |
| PB6 medium | 0 | | 5 | + (4) | 6 | + (1) |
| | | | | ++ (1) | | ++ (2) |
| | | | | | | +++ (3) |
| PB6 low | 0 | | 5 | ++ | 6 | + (1) |
| | | | | | | ++ (2) |
| | | | | | | +++ (3) |

+: mild
++: moderate
+++: severe

At the end of day 3 all animals of groups in which CDAD was induced were still alive but several of them were showing signs of severe diarrhea. On day 4, three days after clindamycin administration, the first animals died. At the end of the treatment period, on day 6, all hamsters in the no treatment group had died. Survival was highest in the vancomycin and PB6 high dose treatment groups, where 4 out of 6 had survived (FIG. 5).

On day 7 a decreased average body weight was observed in all groups in which CDAD was induced. There was a clear inverse dose response relation with PB6 concerning this decrease in weight (Table 5). The animals receiving low dose of PB6, on average lost 3 times more weight than the animals which received the high dose. Weight loss was least with vancomycin treatment. The average body weight in the two groups in which CDAD was not induced had slightly increased over the same time period.

TABLE 5
Average body weight (g) and body weight gain (%).

| | | | Average body weight |
|---|---|---|---|
| | Average body weight (g) | | difference |
| Treatment | Day 0 | Day 7 | (%) day 0 to 7 |
| CDAD Induced | | | |
| No treatment | 219.0 ± 5.1 | * | * |
| | (n = 6) | | |
| Vancomycin | 218.0 ± 4.6 | 215.8^{b} ± 1.7 | −1.7^{b} ± 1.8 |
| | (n = 6) | (n = 4) | |
| PB6 high | 218.8 ± 4.0 | 206.5^{c} ± 5.7 | −5.0^{b} ± 2.1 |
| | (n = 6) | (n = 4) | |
| PB6 medium | 217.8 ± 3.2 | 189.0^{d} ± 2.7 | −12.9^{c} ± 0.7 |
| | (n = 6) | (n = 3) | |
| PB6 low | 217.2 ± 4.5 | 176.5^{e} ± 2.1 | −17.9^{d} ± 1.2 |
| | (n = 6) | (n = 2) | |
| No CDAD | | | |
| induced | | | |
| PB6 high | 217.8 ± 2.3 | 230.0^{a} ± 7.6 | 5.6^{a} ± 3.1 |
| | (n = 6) | (n = 6) | |
| PB6 high | 219.2 ± 3.9 | 225.8^{a} ± 4.8 | 3.1^{a} ± 3.2 |
| | (n = 6) | (n = 6) | |

* no data, all animals had died
^{a,b,c,d,e}values within the same column not baring a common superscript are significantly different (P < 0.05, Least Significant Difference).

The presence of clostridium toxin A and B in fecal samples of the animals in which CDAD was induced, was checked on days 1, 2 and 7. As expected, groups showing high mortality and a high decrease of average body weight also had a high percentage of animals tested positive for these toxins with again an inverse dose response relation with PB6.

TABLE 6
Number of animals tested positive for the presence of C difficile
toxins A or B in their faeces.

| | Day 1 | | Day 2 | | Day 7 | |
|---|---|---|---|---|---|---|
| Treatment | Positive | Tested | Positive | Tested | Positive | Tested |
| No | 3 | 6 | 5 | 6 | * | * |
| treatment | | | | | | |
| Vancomycin | 0 | 6 | 2 | 6 | 1 | 4 |
| PB6 high | 0 | 6 | 3 | 6 | 2 | 4 |
| PB6 | 0 | 6 | 3 | 6 | 2 | 3 |
| medium | | | | | | |
| PB6 low | 0 | 6 | 5 | 6 | 2 | 2 |

no data, all animals had died

Conclusion

This study was designed and conducted to evaluate the efficacy of B. PB6 in treating CDAD in the hamster model of clindamycin-induced CDAD, a well established and highly sensitive model for this infection. Hamsters that were not treated after induction of CDAD all got severe diarrhea, lost weight and died within five days. Treatment with PB6 resulted in a dose related response. Diarrhea, body weight reduction and mortality were least with the highest dose of PB6. At the stop of treatment the highest dose of PB6 showed to have been equally efficient as vancomycin in helping hamsters to survive clindamycin-induced CDAD.

Evaluation of the Efficacious Components of the Orally Administered Bacillus PB6

The new dietary concept of probiotics (such as inulin) is gaining popularity among nutritionists, physicians, food manufactures and consumers. The most widely accepted definition of a probiotic food is: “A probiotic is a selectively fermented ingredient that allows specific changes, both in the composition and activity of the gastrointestinal microflora that confers benefits upon the host well-being and health.” To be classified as a probiotic ingredient in food products, that ingredient must 1) resist digestion (hydrolysis) by alimentary enzymes in the stomach and small intestine; 2) enter the colon chemically intact and subsequently undergo partial or complete fermentation; and 3) stimulate active growth and/or activity of health-stimulating intestinal bacteria. Health benefits of probiotic-stimulated bacteria growth are wide-ranging and include such benefits as positive effects on colon cancer and pathogens, decreased triacylglycerols, increased absorption of Ca and Mg, and increased stool frequency and fecal weight. Inulin is a polydisperse β(2→1) fructan with chain lengths ranging from 2 to 60 units. The unique linkage between the fructose molecules of inulin and oligofructose distinguish them from typical carbohydrates. They resist digestion by human alimentary enzymes and absorption in the small intestine but are hydrolyzed and fermented by colonic microflora and therefore classified as a probiotic dietary fiber.^{42,43,44,45,46}

The fact that probiotics stimulate the active growth and/or activity of health stimulating intestinal bacteria, make them very interesting products to be administered together with probiotics such as Bacillus PB6.

In addition, Bacillus amyloliquefaciens PB6 does not inhibit the growth of “healthy” bacteria from the gut flora such as LactoBacillus spp. and Bifidobacterium spp.

Materials and Methods

Identification of Isolated PB6 Bacteria

The PB6 Bacillus strain isolated is a Gram-positive rod and possesses catalase. Malachite Green staining confirmed that this microorganism possesses endospores and is a spore-former. This microorganism is also a “swarmer” as upon extended incubation period, it tends to spread over the entire agar surface. In terms of relatedness to known Bacillus spp., the Bacillus strain isolated has 92.0% ID. Based on the API biochemical profiles, the PB6 Bacillus strain was putatively identified as B. subtilis. The PB6 Bacillus strain was therefore deposited as ATCC—PTA 6737 and named there Bacillus subtilis. U.S. patent application Ser. No. 10/306,365, filed Nov. 27, 2002, is incorporated herein by this reference.

16 rRNA Gene Sequencing

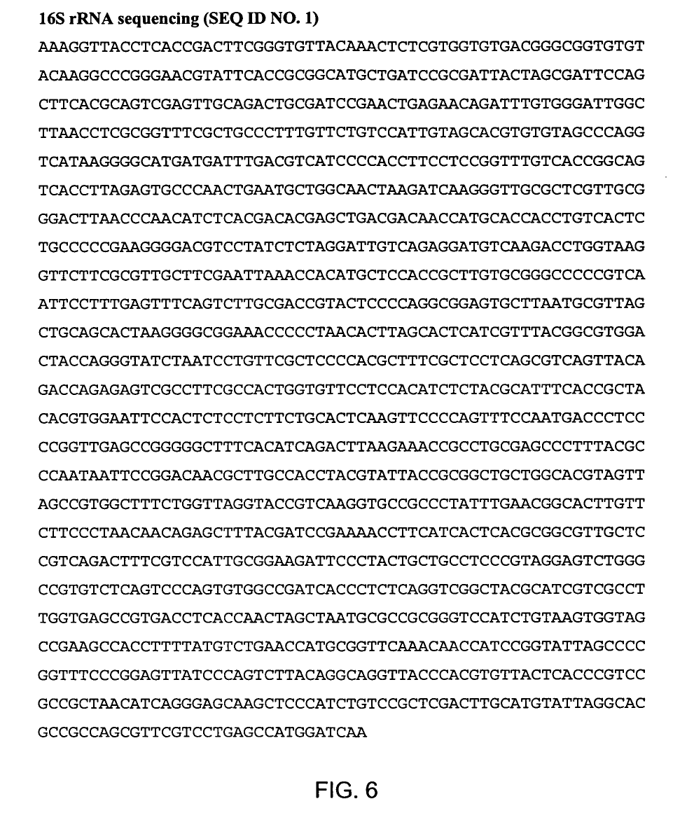

The nearly complete 16 rRNA sequence of Bacillus PB6 was completed and aligned (SEQ ID NO:1, FIG. 6).

GyrA Sequencing

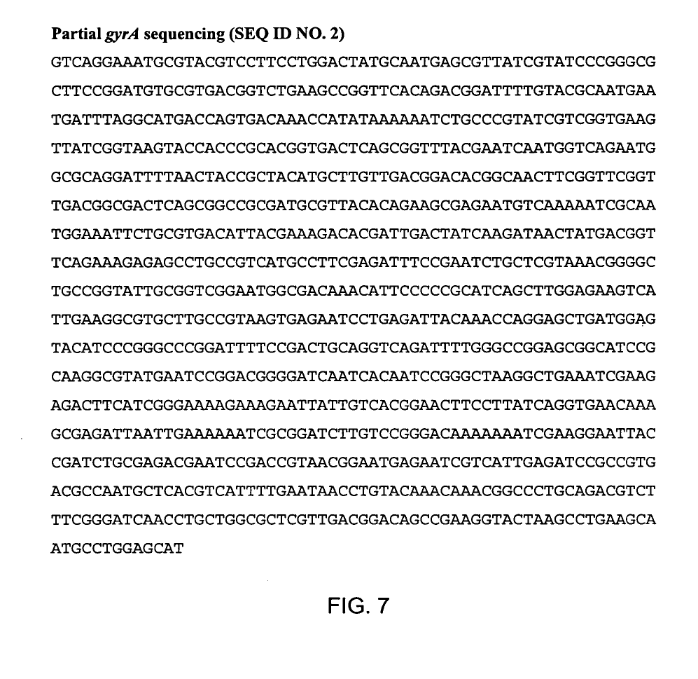

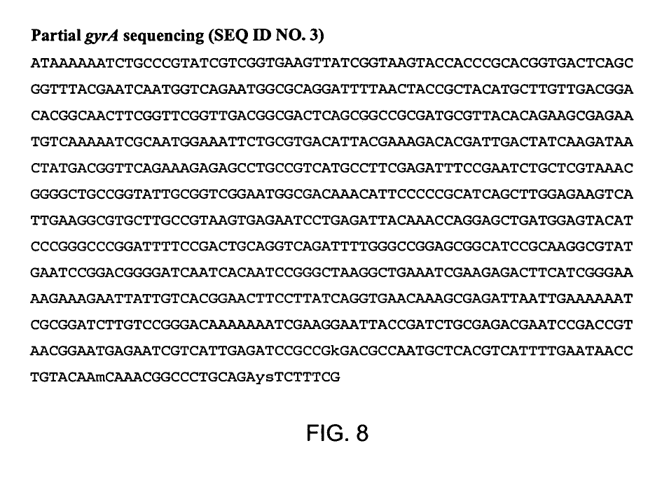

Partial gyrA sequences of PB6 obtained by two different groups are shown in FIG. 7 (SEQ ID No. 2) and FIG. 8 (SEQ ID NO. 3).

DNA:DNA Hybridizations

Hybridizations were performed under stringent conditions (at 40° C.) according to a modification of the method described by Ezaki et al.⁴⁷ The DNA homology percentages are the mean of minimum 4 hybridizations. The value given between brackets is the difference between the reciprocal values. With this technique, the average standard deviation is 14 units.⁴⁸ The results are presented in Table 7.

TABLE 7
% DNA homology

| | % DNA homology | | |
|---|---|---|---|
| Bacillus PB6 | 100 | | |
| B. amyloliquefaciens LMG | 75 (13) | 100 | |
| 9814^{T} | | | |
| B. velezensis LMG 22478^{T} | 90 (6) | 80 (8) | 100 |

A DNA homology above 70%, the generally accepted limit for species delineation,⁴⁹ is found between Bacillus PB6, LMG 9814^{T} and LMG 22478^{T}.

From these results, it appears that B. velezensis and B. amyloliquefaciens belong to the same genospecies and are therefore subjective synonyms such that in applying nomenclatural rule 42 the oldest legitimate name should be retained, i.e. B. amyloliquefaciens; and that Bacillus PB6 (ATCC-PTA 6737) may more properly be categorized to the species B. amyloliquefaciens.

Testing of Antagonistic Properties of Bacillus Amyloliquefaciens by the Streak-Line Assay

Bacillus amyloliquefaciens PB6 was inoculated as a straight line on Tryptone Soy blood plates (Oxoid, Belgium), after 24 hours of incubation at 37° C. in aerobic conditions, the different indicator strains were inoculated perpendicularly to the Bacillus PB6 culture. Plates inoculated with Clostridia species were incubated in anaerobic conditions using Anaerogen Pak (Oxoid, Belgium). After overnight incubation at 37° C., antagonistic effects were evaluated by the appearance of clear zones surrounding the junctions of the streak-lines indicating the inhibitory effects of one organism against the other.

Extraction and Antimicrobial Screening of the Secondary Metabolites of Bacillus Amyloliquefaciens PB6.

Bacillus amyloliquefaciens PB6 was grown on Trypton Soya Agar plates supplemented with 5% sheep blood (Oxoid, Belgium) for 24 hours at 37° C. This culture was used to inoculate 100 ml Tryptic Soy broth supplemented with 0.6% yeast extract (Oxoid, Belgium). After incubation for 24 hours in a shaking incubator (100 rpm) at 37° C., the broth was mixed (3 times) with equal amounts of diethyl ether (Acros, Belgium). After extraction of the metabolites, both layers were separated and the ether fraction was collected and centrifuged at 4000 rpm for 5 min.

Afterwards, the solvent was removed in vacuo using a rotating evaporator. The residue was weighed and dissolved in dimethylsulfoxide (Acros, Belgium) resulting in a final concentration of 10000 μg/ml crude extract. Further dilutions (500, 250, 100, 50, 25 and 10 μg/ml) were made in a mixture of DMSO/water with ratio 1/11. Finally, 25 μl of each dilution was pipetted into the wells of the microtiter plates (Labsystems, Finland).

The bacterial strains Clostridium perfringens ATCC13124 (C1600L, Oxoid, Belgium) and Clostridium difficile ATCC9689 (C1610L, Oxoid, Belgium) were purchased as freeze-dried culti-loops and brought into culture according to the manufacturer's instructions. From both cultures a McFarland standard (A₆₂₅ₙₘ=0.100) was prepared in Anaerobic Basal broth (Oxoid, Belgium). 250 μl of this standard was added to 10 ml of fresh Anaerobic Basal broth, and 225 μl of this medium was pipetted into the wells. Yielding a final cell density of 5×10⁵ cfu/ml in each well. The microtiter plates were incubated in anaerobic conditions for 18 hours (C. perfringens) and 48 hours (C. difficile) using Anaerogen Compact (Oxoid, Belgium) in an airtight plastic bag. Before and after the incubation period, optical density (OD) of each well was measured using the Bioscreen C analyzer (Labsystems, Finland). White light (Wide Band) was used to measure the OD. Tests were done in duplicate and also control of the medium, medium plus inoculum (negative control) and a positive control; vancomycin (Fluka, Belgium) at three concentrations (0.1, 0.5 and 1.0 μg/ml) was included in the test batch. Minimum inhibition concentration (MIC) was defined as the lowest concentration where no growth occurred or where no increase of OD was detected.

Determination of the Enhancing Effect of a Co-Factor on the Activity of Surfactin

The ether extract of the B. amyloliquefaciens PB6 fermentation product was separated by preparative TLC (Kieselgel 60, 20×20 cm, 2 mm layer thickness). The eluent used was hexane/acetone (30/70). The surfactins were isolated in the zone with Rf 0 to 0.33. The MIC against C. perfringens of the surfactins isolated from this zone was between 10 and 25 μg/ml. In another zone (Rf 0.33 to 0.76) one or more products were found that were not active against C. perfringens (MIC>100 μg/ml). When a combination was tested of the products of both zones, with a final concentration of both extracts of 10 μg/ml each, there was no growth of C. perfringens. The MIC of surfactins decreased to below 10 μg/ml when combined with these products that were not active on their own.

Determination of the Effect of Acidic Treatment on the Antimicrobial Activity of Surfactin

A solution was prepared of 750 μg/ml surfactin (Sigma) in acetonitrile/0.1 N HCl (1/1; v/v). 0.5 ml of this solution was incubated at 37° C. for 30′, 60′ or 90′, after which 0.25 ml of a 0.1 N NaHCO₃ solution was added and the mixture was mixed using a vortex shaker. All solutions were screened for activity against Clostridium perfringens.

Determination of Enterotoxin Production by Bacillus Amyloliquefaciens PB6

Bacterial strains and culture conditions. Bacillus amyloliquefaciens PB6 was grown in 100-ml volume of Tryptic Soy Broth (Becton Dickenson and Company, Cockeysville, Md.) supplemented with 0.6% yeast extract (Oxoid Limited, England) (TSBYE) and incubated at 37° C. in a shaker incubator set at 100 rpm. One toxin-producing strain of B. cereus ATCC 11778 was also grown in TSBYE at 37° C. in a shaker incubator. Similarly, non toxin-producing strains of B. cereus ATCC 49064 and Escherichia coli ATCC 25922 were also grown in TSBYE at 37° C. under aerobic conditions. All bacterial strains used in this study were transferred weekly to fresh TSBYE and then kept in a 4° C. refrigerator as working culture. Freshly grown strains were re-suspended in 40% glycerol and kept at −80° C. freezer for long-term storage.

Enterotoxin production. A 1-ml volume of overnight test culture was inoculated into 50 ml of Brain Heart Infusion (BHI) supplemented with 1% glucose (BHIG) and incubated in a shaking incubator (100 rpm) for 6 h at 32° C.³¹. Bacterial cells were precipitated by centrifugation at 5000×g for 10 min and the supernatant was collected for cytotoxicity studies of Vero cells³¹. The proteins in the supernatant were then concentrated ten-fold using up to 80% saturated ammonium sulfate solution (561 g per liter)³³. After centrifugation at 10000×g for 20 min, the supernatant was decanted and protein-pellet was re-suspended in 2.5 ml of phosphate buffer (20 mM; pH 6.8). Residual salts of ammonium sulfate were removed by dialysis against the same buffer at 4° C. for 6 h. The final volume of the dialyzed protein solution was adjusted to one-tenth of the original volume (5 ml) using the phosphate buffer (20 mM; pH 6.8)³¹.

Emetic toxin production. A 1-ml volume of overnight test culture was inoculated into 50 ml of Brain Heart Infusion (BHI) supplemented with 1% glucose (BHIG) and incubated for 6 h at 32° C. in a shaker incubator set at 250 rpm. Bacterial cells were precipitated by centrifugation at 2000×g for 10 min at 4° C.³¹. The supernatant was collected and then autoclaved at 121° C. for 15 min to remove heat-labile enterotoxins²⁰. The heat-treated filtrate with possibly the heat-stable emetic toxin was collected for the vacuolation assay on HEp-2 cells²⁰.

Preparation of Vero and HEp-2 cells. African green monkey kidney cells (Vero) or HEp-2 (human carcinoma of the larynx) were maintained as monolayer cultures in 30 ml of Medium 199 with Earle's modified salts (MEM) containing 2 mM L-glutamine, 10 mM sodium bicarbonate, 1% non-essential amino acids, 100 UI/ml penicillin, 0.1 mg/ml streptomycin, and 3 ml fetal calf serum (10%). The leucine-free medium (MEM) was prepared on the basis of minimum essential medium (Gibco), which also contained 1.8 mM CaCl₂, 0.4 mM MgCl₂, 5.0 mM KCl, 0.12 M NaCl, 3.2 mM NaH₂PO₄, and 20 mM Hepes (pH 7.7). The Vero or HEp-2 cells were incubated in MEM at 5% CO₂ at 37° C. Confluent monolayer cultures of Vero or HEp-2 cells were sub-cultured by discarding the media before washing with 5 ml of PBS (pH 7.7). Vero or HEp-2 cells were then detached from the culture flask by the addition of 2 ml of trypsin solution (0.25% trypsin; 0.025% EDTA). The levels of loosen Vero or HEp-2 cells were determined microscopically before the addition of MEM medium (8 ml) to prevent further effect of trypsin. A 5-ml aliquot of freshly trypsinised Vero or HEp-2 cells were then transferred to new culture flask containing 15 ml of MEM for incubation at 37° C. under 5% CO₂.

TECRA® Bacillus Diarrheal Enterotoxin (BDE) Visual Immunoassay. All components of the test kit (TECRA International Pte Ltd, Chatswood, NSW, Australia) were kept at 20-25° C. prior to testing of samples for BDE. As stated by the manufacturer, microtiter wells containing high affinity antibodies specific for BDE were pre-soaked with wash solution, provided in the kit and allowed to stand for 10 min at 20-25° C. The wells were emptied before aliquots containing 200-μl volume of test samples and controls (positive and negative) were transferred into individual wells. The wells were incubated at 37° C. for 2 h. The wells were washed 4 times before an aliquot of 200 μl of conjugate was added to each well and incubated at 25° C. for 1 h. Each well was washed 5 times before 200 μl of substrate was added to each well and incubated at 25° C. for 30 min. After 30 min, the calorimetric development for each well was compared against the TECRA Color Card provided in the test kit.

Oxoid Bacillus cereus Enterotoxin Reversed Passive Latex Agglutination (BCET-RPLA). The test was developed for the detection of diarrheal enterotoxin of Bacillus cereus by reversed passive agglutination (RPLA) (Unipath, Basingstoke, UK). Polystyrene latex particles are sensitized with purified antiserum from rabbits immunized with diarrheal enterotoxin from B. cereus. The kit also provided the both positive (enterotoxin) and negative (latex particles without specific B. cereus anti-enterotoxin) controls. Aliquots containing 25 μl of diluent and test or control (positive and negative) samples were dispensed successively into 2 different sets of V-well microtiter plates. Solutions containing 25 μl of sensitized latex and latex control were then dispensed into the first and second set of V-well microtiter plates, respectively. Each well was examined for agglutination after 20-24 h of incubation at 25° C.

Measurement of cytotoxicity. Freshly trypsinised Vero cells were re-suspended in 30 ml of leucine-free medium (MEM). One milliliter of the Vero cell suspension was transferred to each of the 24 wells (˜5×10⁴ cells per well). Cells were washed once with 1 ml of leucine-free MEM and incubated for 2 h at 37° C. under 5% CO₂. After 2 h, the growth medium was removed and each well was washed once with 1 ml of leucine-free MEM. A 1-ml volume of preheated (37° C.) leucine-free MEM was added to each well, followed by 50 μl of supernatant or filtrate of Bacillus amyloliquefaciens PB6, B. cereus ATCC 11778, B. cereus ATCC 49064 and E. coli ATCC 25922 immediately thereafter. The inoculated wells were incubated at 37° C. with 5% CO₂ for 2 h. After 2 h of incubation, supernatant or filtrate-treated Vero cells were washed once with 1 ml of pre-heated (37° C.) leucine-free MEM. A volume of 3001 of solution containing radioactive-labeled isotope (16 μl ¹⁴C-leucine in 8 ml of leucine-free MEM) (Perkin Elmer Asia, Singapore) was added to each of the well. ¹⁴C-leucine labeled Vero cells were incubated at 37° C. without CO₂ for 1 h and thereafter the growth medium was discarded. Subsequently, 1-ml aliquots of solution containing 5% trichloroacetic acid were added to each well containing ¹⁴C-leucine labeled Vero cells before incubating at 25° C. for 10 min. After 10 min of incubation, the contents in each well were washed twice with 1 ml of 5% trichloroacetic acid-solution. A 300-μl volume of 0.1 M KOH was then added to each well and incubated at 25° C. for another 10 min. After 10 min of incubation, 2-ml volume of scintillation liquid was added to each well. Finally, all contents from each well were transferred into scintillation tubes. All scintillation tubes were agitated for 1 min before performing radioactivity counts. The percent inhibition of ¹⁴C leucine uptake by Vero cells is calculated using the following formula.

Percent inhibition of ¹⁴C leucine uptake=[(cpm for Vero cells without toxin added−cpm for test sample)]/[cpm for Vero cells without toxin added]×100%. The cpm used for calculating percent inhibition of ¹⁴C leucine uptake by Vero cells has to be subtracted by the value for background counts (˜30-60 cpm). No toxin is present if the inhibition of ¹⁴C leucine uptake is less than 20% after the ten-fold concentration. Each assay was conducted in duplicates. The results are presented in Tables 8 and 9.

TABLE 8
Effect of bacterial filtrate on Vero and HEp-2 Cell Lines

| | |
|---|---|
| Vero cells | HEp-2 cells |
| Cytotoxic | Vacuole |
| effect | response |

| Bacteria | 1 h | 24 h | 6 h | 24 h |
|---|---|---|---|---|
| Bacillus amyloliquefaciens | −^{a} | − | − | − |
| PB6 | | | | |
| Escherichia coli ATCC 25922 | − | − | − | − |
| Bacillus cereus ATCC 11778 | − | − | − | − |
| Bacillus cereus ATCC 49064 | ++^{b} | ++++ | − | +++ |

^{a}“−” indicates no cytotoxic effect or presence of vacuoles observed.
^{b}“+” indicates destruction of Vero cell or vacuole production in HEp-2 cells have occurred.

TABLE 9
% inhibition of ¹⁴C-leucine isotope uptake

| Concentrated filtrates of bacteria | % inhibition^{a} |
|---|---|
| Bacillus amyloliquefaciens PB6^{b} | 1 |
| Escherichia coli ATCC 25922^{c} | 1 |
| Bacillus cereus ATCC 11778^{d} | 17 |
| Bacillus cereus ATCC 49064^{e} | 100 |

^{a}Toxin from the tested strain is considered negative if the inhibition of ¹⁴C leucine uptake is less than 20% after ten-fold concentration. (SCAN report).
^{b}Test microorganisms,
^{c}negative and ^{e}positive and controls and
^{d}non-toxin producing strain.

HEp-2 Cells Vacuolation Assay. A 25-μl volume containing filtrates from test and control cultures were serially diluted (2-fold) in 0.15 M NaCl solution and dispensed across wells of a 96-well tissue culture plate (Gibco Ltd, Uxbridge, UK). Volumes containing 100 μl of freshly trypsinized HEp-2 cells were added to each well and incubated for 24 h at 37° C. All vacuolation assays were performed in duplicates. Microscopic examination for the presence of vacuole formation in HEp-2 cells was conducted at the 6^{th} and 24^{th} hour-intervals.

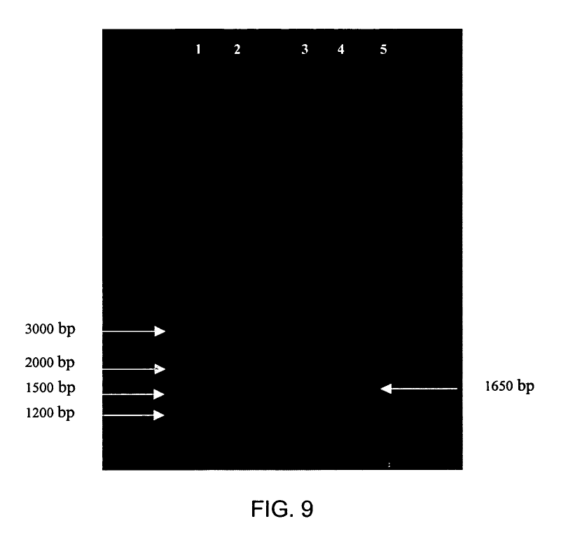

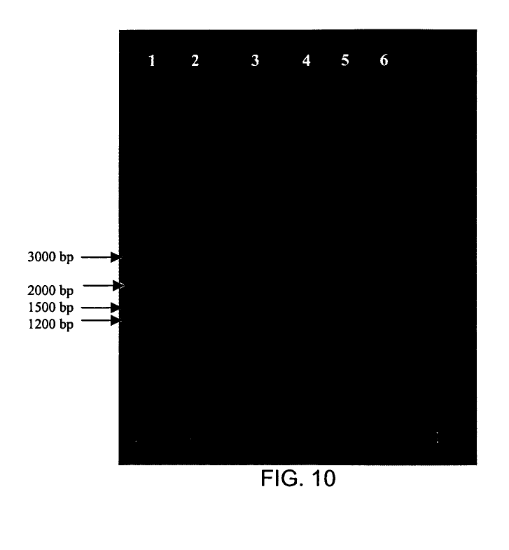

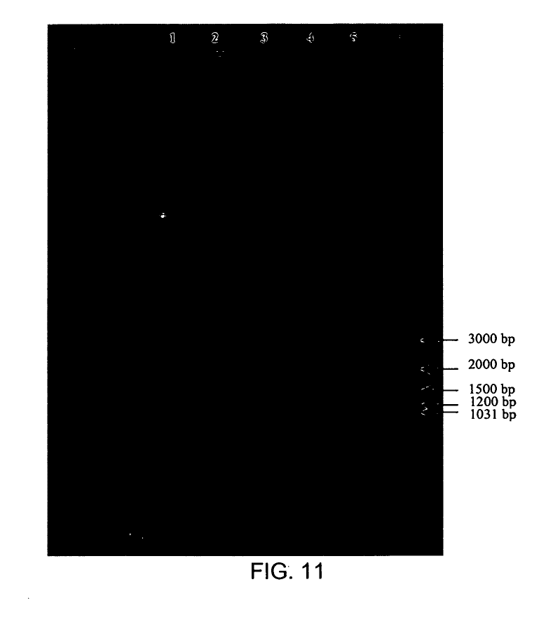

PCR-based methods. PCR primers were developed for the non-haemolytic enterotoxin (NheB-nheC)^{25,26}, hemolysin BL (HblD-hblA)^{10,11}, and enterotoxin K (EntK)^{27,28}. The sequences of the NheB-nheC primers were 5′CGGTTCATC-TGTTGCGACAGC 3′ and 3′GTCCTCGTGTTCGTCTTC-AGC 5′. The primer sequences for HblD-hblA were 5′CGCT-CAAGAACAAAAAGTAGG 3′ and 3′ TCCCTAATGT-CTAAATGTTCCTC 5′. The forward and reverse primers for EntK were 5′GAATTACGTTGGCGAATC3′ and 3′CGGG-CGGATGGGA 5′, respectively. Both sample PCR and positive control tubes containing DNA of Bacillus amyloliquefaciens PB6 and toxigenic strains of B. cereus ATCC 11778 and B. cereus ATCC 49064 were placed into a Perkin-Elmer thermal cycler (GeneAmp PCR System 9600, Perkin-Elmer Corp., Norwalk, Conn.) with initial set point temperature of 90° C. The conditions for thermal cycling were 1 cycle at 94° C. for 2 min followed by a 38-cycle temperature cycling routine of 94° C. for 15 s, and 70° C. for 3 min. Following amplification, a final extension at 72° C. for 7 min was performed. The PCR amplification step required approximately 3 h to complete. Aliquots (15 μl) of each reaction product and 15 μl DNA standard ladder (500, 1031, 2000 and 3000 base-pairs) were then electrophoresed at 180 volts for 1 h through a 2% agarose gel (Life Technologies Inc., Gaithersburg, Md.) containing 0.1 μg/ml ethidium bromide (Sigma Chemical Co., St. Louis, Mo.). After electrophoresis, all gels were viewed using a UV-transilluminator (wavelength-302 nm) (UVP Model White/2 UV; UVP Inc., Upland, Calif.) and then photographed using a Polaroid MP-4 Camera (Polaroid Corp., Cambridge, Mass.)(See FIG. 9, FIG. 10 and FIG. 11).

### Example 2

### Determination of the Antimicrobial Properties of Metabolites of Different Probiotics and Bacillus Amyloliquefaciens PB6 Against Clostridium Perfringens and Clostridium Difficile Via Broth Microdilution Method

In this study we investigated the anticlostridial properties of the metabolites from Bacillus PB6 and four commercially available probiotics: Bactisubtil® (Sanofi-synthelabo), Perenterol® (Biodiphar), Bioplus 2B (Miavit GmbH) and Biosporinum (Dniprofarm). Small-scale fermentations were setup with the species isolated from the different probiotics. Ether extracts of the fermentation broth, containing the metabolites, were screened against C. perfringens ATCC13124 and C. difficile ATCC9689 using broth microdilution method. The metabolites of B. PB6 showed significant anticlostridial properties. Minimum inhibition concentrations were situated between 2.5 and 5.0 μg/ml towards C. perfringens and between 5.0 and 10.0 μg/ml towards C. difficile. The ether extracts of the fermentation from Bactisubtil®, Perenterol®, Bioplus 2B and Biosporinum did not have any significant antibacterial activity against both clostridia species.

Although Clostridium species are ubiquitous in nature, their principle habitats are the soil and the intestinal tracts of many animals and humans. The widespread occurrence of C. perfringens, including its spores, in soil samples almost guarantees the frequent presence of this organism on surfaces exposed to dust contamination, including many food items.⁵⁰ C. perfringens is also the species most commonly isolated from human clinical specimens, excluding faeces. It is encountered in a wide variety of clinical settings ranging from simple contamination of wounds to traumatic myonecrosis, intra-abdominal sepsis, intravascular haemolysis, aspiration pneumonia, necrotising pneumonia etc.⁵¹

C. difficile is a major cause of antibiotic-associated diarrhea (AAD) and is also the most frequently identified cause of hospital-acquired diarrhea. In C. difficile-associated disease (CDAD), the primary initiating event involves the disruption of the protective intestinal flora during treatment with antibiotics. As the level of antibiotic drops below inhibitory concentrations, nosocomial pathogens such as C. difficile are able to grow. Colonization occurs by the faecal-oral route, ingested spores survive the gastric acid barrier and start to germinate in the colon.^{52,53} Toxigenic as well as nontoxigenic isolates are capable of forming spores and existing in the hospital environment. As a result, either type can infect the colon and utilize the nutrients that are available because the lack of competition by the normal flora. Whether the organism attaches to the colonic wall is not clear, but it is more likely that the organism grows throughout the lumen of the colon. Toxigenic strains produce and release toxins A and/or B as the cells grow and lyse. This activity, along with the inflammatory response, result in the histopathological events leading to C. difficile-associated diarrhea and colitis.^{54,55,56}

A “probiotic” by generally accepted definition, is a “live microbial” feed or food supplement which beneficially affects the host by improving its intestinal microbial balance. But how does a probiotic work? The effect of probiotics on the intestinal ecosystem impacts in some beneficial way on the consumer. A number of potential benefits arising from changes to the intestinal milieu through probiotics have been proposed, including: increased resistance to infectious diseases, particularly of the intestine, decreased duration of diarrhea, reduction in blood pressure, reduction in serum cholesterol concentration, reduction in allergy etc.⁵⁷

The comparative study described in this paper was set up in order to compare the antimicrobial properties of different probiotic fermentation extracts towards clostridia species

Methods and Materials

Bacillus PB6 and the Bacillus cereus strain isolated from Bactisubtil® (Sanofi-synthelabo) were grown on Trypton Soya Agar plates supplemented with 5% sheep blood (Oxoid, Belgium) for 24 hours at 37° C. The feed probiotic Bioplus 2B contained two different species, Bacillus licheniformis DSM5749 and Bacillus subtilis DSM5750. Both species were also grown on Trypton Soya Agar plates supplemented with 5% sheep blood (Oxoid, Belgium) for 24 hours at 37° C. A mixture of these cultures was used to inoculate 100 ml Tryptic Soy broth supplemented with 0.6% yeast extract (Oxoid, Belgium). Biosporinum, also contains two species, i.e. Bacillus licheniformis and Bacillus subtilis. This product is marketed as lyophilised cultures in glass vials. The content of one vial was resuspended in broth, this mixture was used to inoculate 100 ml Tryptic Soy broth supplemented with 0.6% yeast extract (Oxoid, Belgium). After incubation of all probiotics for 24 hours in a shaking incubator (100 rpm) at 37° C., the broth was mixed (3 times) with equal amounts of diethyl ether (Acros, Belgium). After extraction of the metabolites, both layers were separated and the ether fraction was collected and centrifuged at 4000 rpm for 5 min.

Afterwards, the solvent was removed in vacuo using a rotating evaporator. The residue was weighed and dissolved in dimethylsulfoxide (Acros, Belgium) resulting in a final concentration of 10000 μg/ml crude extract. Further dilutions (500, 250, 100, 50, 25 and 10 μg/ml) were made in a mixture of DMSO/water with ratio 1/12. Finally, 25 μl of each dilution was pipetted into the wells of the microtiter plates (Labsystems, Finland). Saccharomyces boulardii used in Perenterol® (Biodiphar) was grown on Sabouraud dextrose agar (Oxoid, Belgium) for 48 hours at 37° C. This culture was used to inoculate 100 ml Sabouraud liquid medium (Oxoid, Belgium). After incubation for 2 days in a shaking incubator (100 rpm) at 37° C., the same procedure was followed to extract the metabolites.

The bacterial strains Clostridium perfringens ATCC13124 (C1600L, Oxoid, Belgium) and Clostridium difficile ATCC9689 (C1610L, Oxoid, Belgium) were purchased as freeze-dried culti-loops and brought into culture according to the manufacturer's instructions. From both cultures a McFarland standard (A₆₂₅ₙₘ=0.100) was prepared in Anaerobic Basal broth (Oxoid, Belgium). 250 μl of this standard was added to 10 ml of fresh Anaerobic Basal broth, and 225 μl of this medium was pipetted into the wells. Yielding a final cell density of 5×10⁵ cfu/ml in each well. The microtiter plates were incubated in anaerobic conditions for 18 hours (C. perfringens) and 48 hours (C. difficile) using Anaerogen Compact (Oxoid, Belgium) in an airtight plastic bag. Before and after the incubation period, optical density (OD) of each well was measured using the Bioscreen C analyser (Labsystems, Finland). White light (Wide Band) was used to measure the OD. Tests were done in duplo and also control of the medium, medium plus inoculum (negative control) and a positive control; vancomycin (Fluka, Belgium) at three concentrations (0.1, 0.5 and 1.0 g/ml) was included in the test batch. Minimum inhibition concentration (MIC) was defined as the lowest concentration where no growth occurred or where no increase of OD was detected.

Results and Discussion

Strong increase of OD was noticed in the negative control wells for both species. No growth occurred in the wells containing 1.0 and 0.5 μg/ml vancomycin, 0.1 μg/ml vancomycin did not inhibit the growth of C. perfringens. In the wells containing 1 and 2.5 μg/ml PB6-extract, normal growth of C. perfringens occurred. From 5 μg/ml on, no growth of C. perfringens could be noticed. MIC of the PB6 crude fermentation extract against C. perfringens was between 2.5 and 5.0 μg/ml. Also for C. difficile a strong increase of OD was noticed in the negative control wells. No growth occurred in the wells containing 1.0 and 0.5 μg/ml vancomycin, 0.1 μg/ml of vancomycin did not inhibit the growth. In the wells containing 1, 2.5 and 5 μg/ml crude PB6-extract, normal growth of C. difficile occurred. From 10 μg/ml on, no growth could be detected. MIC of the ether extract of PB6 metabolites against C. difficile was between 5.0 and 10 μg/ml. The ether extracts from Bactisubtil® (B. cereus), Perenterol® (S. boulardii), Bioplus 2B (Bacillus licheniformis DSM5749 and Bacillus subtilis DSM5750) and Biosporinum (Bacillus licheniformis sp. and Bacillus subtilis sp.) did not have any significant antibacterial activity for all concentrations tested. MIC was above 50 μg/ml for both clostridia species tested. These results can be found in Table 10.

TABLE 10
Results of the screening with the crude extracts against C. perfringens and
C. difficile

| | MIC (μg/ml) | | | | |
|---|---|---|---|---|---|
| | Bacillus PB6 | Bactisubtil ® | Perenterol ® | Bioplus 2B | Biosporinum |
| C. perfringens | 2.5-5 | >50 | >50 | >50 | >50 |
| ATCC13124 | | | | | |
| C. difficile | 5-10 | >50 | >50 | >50 | >50 |
| ATCC9689 | | | | | |

Conclusion

We investigated the anticlostridial properties of ether extracts from B. PB6, Bactisubtil® (B. cereus), Perenterol® (S. boulardii), Bioplus 2B (B. licheniformis DSM5749 and B. subtilis DSM5750) and Biosporinum (Bacillus licheniformis sp. and Bacillus subtilis sp.). Lab-scale fermentations with these species were set up and the ether extracts were screened against C. perfringens ATCC 13124 and C. difficile ATCC9689 using broth microdilution techniques. The metabolites of B. PB6 possess strong anticlostridial properties, MIC are situated between 2.5 and 5.0 μg/ml towards C. perfringens and between 5.0 and 10.0 μg/ml towards C. difficile. The ether extracts from Bactisubtil®, Perenterol®, Bioplus 2B and Biosporinum fermentations did not have any significant effect against C. perfringens ATCC13124 and C. difficile ATCC9689.

### Example 3

### Efficacy of Bacillus PB6 Against IBD

It is known that surfactin inhibits the activity of cytosolic PLA₂, an enzyme centrally involved in many inflammatory processes.⁵⁸ The inhibition of inflammatory processes makes surfactin producing probiotics very interesting for the treatment of inflammatory diseases, such as Inflammatory Bowel Disease (IBD).

Inflammatory bowel disease refers to two chronic diseases that cause inflammation of the intestines: ulcerative colitis (UC) and Crohn's disease (CD). Although the diseases have some features in common, there are some important differences.

CD is a chronic inflammation of the intestinal wall, typically affecting the full thickness of the intestinal wall. Most commonly, it occurs in the lowest portion of the small intestine (ileum) and the large intestine, but it can occur in any part of the digestive tract from the mouth to the anus and the skin around the anus.

In recent decades, CD has become more common both in western and developing countries. It occurs roughly equally in both sexes, and is more common among Jewish people. Most cases begin before the age of 30; the majority starts between the ages of 14 and 24.

In each person, the disease affects specific areas of the intestine, sometimes with normal areas (skip areas) sandwiched between the affected zones. In about 35% of CD sufferers, only the ileum is affected. In about 20%, only the large intestine is affected. In about 45% of patients, both the ileum and the large intestine are affected.

The causes of CD are unknown. Research has focused on three main possibilities: a dysfunction of the immune system, infection, and diet.

The most common early symptoms of CD are chronic diarrhea, abdominal pain, fever, loss of appetite, and weight loss. Symptoms differ among CD patients, but there are four common patterns:
inflammation with pain and tenderness in the right lower part of the abdomen;
recurring acute intestinal obstructions that cause severe painful spasms of the intestinal wall, swelling of the abdomen, constipation, and vomiting;
inflammation and chronic partial intestinal obstruction causing malnutrition and chronic debility;
abnormal fistulas and abscesses that often cause fever, painful masses in the abdomen, and severe weight loss.

UC is a chronic disease in which the large intestine becomes inflamed and ulcerated, leading to episodes of bloody diarrhea, abdominal cramps, and fever. The disease can start at any age, but usually begins between the ages of 15 and 30.

Unlike CD, UC does not usually affect the full thickness of the intestine, and does not affect the small intestine. The disease usually begins in the rectum or the sigmoid colon, and eventually spreads partially or completely through the large intestine. In some patients, most of the large intestine is affected early on.

About 10% of patients who appear to have UC only suffer a single attack. However, a proportion of such patients may actually be suffering from an undetected infection, rather than true UC. For most patients, UC is a chronic disease that waxes and wanes over time. The causes of UC remain unknown. Heredity and over-active immune responses in the intestine are thought to be contributing factors.

Determination of the In Vivo Efficacy of Orally Administered Bacillus PB6 in the Treatment of TNBS-Induced Colitis in Rats (a Model for Colitis in Humans).

In this experiment, the efficacy of PB6 was studied against colitis in rats induced by the rectal administration of 2,4,5-trinitrobenzene sulfonic acid (TNBS).

Study Design

Two consecutive trials were done. Male Wistar rats were obtained from the National Centre for Laboratory Animal Sciences (Hyderabad, India) (trial 1) or from the Department of Animal Medicine, TANUVAS (Chemai, India) (trial 2). At the beginning of the treatment period, the animals were 10 to 12 weeks old. Upon their arrival at the test facility, the animals were given a complete clinical examination under the supervision of a veterinarian to ensure that they were in good condition. The animals were acclimatized to the study conditions for a period of at least 7 days. Per trial, animals were randomised and allocated to one of the study groups. The animals were housed per study group in polycarbonate cages (421×290×190 mm, L×W×H). The animal room and test room conditions were set as follows: temperature: 22±3° C., relative humidity: 50±20%, light/dark cycle: 12 hr/12 hr (light 07.00-19.00) and ventilation: approximately 7 cycles/hour of filtered, non-recycled air. All animals had free access (except for the overnight fasting prior to TNBS administration) to rat pellet feed from the National Institute of Nutrition (NIN, Hyderabad, India) and Aquaguard purified water ad libitum. In both trials the day of induction of colitis was set as day 1. Colitis was induced, after an overnight fast, using a single intrarectal administration of TNBS at 100 mg/Kg body weight, 8 cm proximal to the anus. The colitis-negative control groups were given saline intrarectally (0.5 ml per animal once) on day 1. Colitis-negative and colitis-positive control groups were given distilled water orally at 10 ml/kg, 3 times daily with 4 h inter dosing, starting on day 1 and up to and including day 7. In the first trial, groups with TNBS-induced colitis were treated with PB6 (1.5 10⁸ CFU/Kg or 1.5 10⁹ CFU/Kg respectively), 3 times daily with 4 h interdosing, starting on day 1 and up to and including day 7; with mesalazine (250 mg/Kg/day), starting on day 1 and up to and including day 7; or with infliximab (3 mg/Kg) as a single dose on day 1. The second trial partially repeated the first one concerning the PB6 (1.5 10⁸ CFU/Kg), mesalazine and infliximab treatments and included an additional treatment with S. boulardii (1.5 10⁸ CFU/Kg), 3 times daily with 4 h interdosing, starting on day 1 and up to and including day 7. The first or only (in case of infliximab) dose of treatment was given within 2 (distilled water, PB6, S. boulardii and mesalazine) or 3 (infliximab) hours after administration of TNBS. Except for infliximab, which was injected intravenously⁵⁹, all treatments were administered by gavage. Twice daily observations were made for clinical signs and mortality. Body weights of animals were recorded on days 1, 4 and 7. On day 8 animals were sacrificed and a 5 cm long segment of the colon (from 10 to 5 cm proximal to the anus) was excised. These segments were opened longitudinally. Contents were removed by washing with saline and gross morphology was scored using the following scale: 0-no ulcers or inflammation, 1-no ulcers only local hyperaemia, 2-ulceration without hyperaemia, 3-ulceration and inflammation at one site only, 4-two or more sites of ulceration and inflammation, and 5-ulceration extending more than 2 cm. The weight of each 5 cm colonic segment was also recorded to assess inflammatory induced edema.

Test Preparations

TNBS 5% (w/v) in water (Sigma-aldrich, St Louis, USA) was diluted to a 2.5% solution with ethanol 50%. Dose volume was 4 ml/Kg body weight. PB6 Dry (Kemin Health, Des Moines, USA), a Bacillus ‘PB6’ fermentation broth dried on a malto- and cyclodextrin carrier, was suspended in distilled water to concentrations of 1.5 10⁷ and 1.5 10⁸ CFU/ml. Dose volume was 10 ml/Kg body weight. Saccharomyces boulardii (Enterol®, Biodiphar, Brussel, Belgium) was suspended in distilled water to a concentration of 1.5 10⁷ CFU/ml. Dose volume was 10 ml/Kg body weight. Mesalazine (Mesacol®, Sun Pharmaceutical Ind. Ltd, Mumbai, India) tablets were powdered using pestle and mortar and a solution in distilled water was prepared containing 25 mg 5-aminosalicylic acid per ml. Dose volume was 10 ml/Kg body weight. Remicade® (Infliximab) (Centocor B. V., Leiden, The Netherlands) was first reconstituted with 10 ml water for injection and was further diluted to 2 mg/ml concentration using saline. Dose volume employed was 1.5 ml/Kg body weight. All body weight dependant doses were administered on the basis of the last individual body weight taken. Fresh preparations were made prior to each administration. The preparations were stirred vigorously before each dosing.

Results and Discussion

In the first trial one of the rats treated with Infliximab died on day 2. This animal had been given a second injection on day 1 because at the first one drug solution oozed out. Some animals showed mild signs of diarrhea after induction of colitis with TNBS. The number of observations of diarrhea recorded (and the number of animals affected) in the different treatment groups from day 1 up to and including day 7 were in the first trial 0, 22 (2), 4 (1), 0, 0 and 8 (1) for colitis-negative control, colitis-positive control, PB6 3×1.5 10⁸ CFU/Kg/day, PB6 3×1.5 10⁹ CFU/Kg/day, mesalazine 250 mg/Kg/day and infliximab 3 mg/Kg single dose respectively; and in the second trial 0, 42 (4), 10 (1), 34 (3), 12 (1) and 24 (2) for colitis-negative control, colitis-positive control, PB6 3×1.5 10⁸ CFU/Kg/day, S. boulardii 3×1.5 10⁸ CFU/Kg/day, mesalazine 250 mg/Kg/day and infliximab 3 mg/Kg single dose respectively. The colitis-positive control groups showed substantial body weight loss accompanying the colitis. In trial 1 this loss was higher than in trial 2, what might be related to a difference in age and growth rate at the moment of induction of colitis. The lower body weight in general at the start and the higher body weight gain in terms of percentage over the trial period of the colitis-negative control group in trial 1 probably indicate that these animals were on average somewhat younger than those in trial 2. While common treatments in trial 1 all significantly suppressed the negative effect of colitis on body weight gain, in trial 2 this was only the case with PB6 treatment (Table 11).

TABLE 11
Average body weight (g) and average body weight gain (%) with their
standard deviation.

| | | | | Av. Body weight |
|---|---|---|---|---|
| | Av. Body weight (g) | | | gain (%) |
| | Day 1 | Day 4 | Day 7 | Day 1 to 7 |
| Trial 1 | | | | |
| Colitis-negative control | 178 ± 17 | 191 ± 13* | 203 ± 13* | 14.3 ± 4.5* |
| Colitis-positive control | 182 ± 15 | 167 ± 16 | 159 ± 18 | −13.0 ± 4.5 |
| PB6 1.5 × 10⁸ CFU/Kg | 180 ± 13 | 186 ± 13 | 195 ± 10* | 8.8 ± 8.0* |
| PB6 1.5 × 10⁹ CFU/Kg | 180 ± 16 | 187 ± 15 | 199 ± 17* | 10.3 ± 1.1* |
| Mesalazine | 181 ± 11 | 187 ± 10 | 196 ± 10* | 8.8 ± 1.4* |
| Infliximab | 180 ± 14 | 183 ± 7 | 186 ± 6* | 1.3 ± 5.4* |
| Trial 2 | | | | |
| Colitis-negative control | 207 ± 20 | 210 ± 19 | 214 ± 17 | 3.5 ± 1.8* |
| Colitis-positive control | 208 ± 21 | 196 ± 21 | 193 ± 19 | −7.5 ± 3.2 |
| PB6 1.5 × 10⁸ CFU/Kg | 206 ± 23 | 206 ± 23 | 212 ± 26 | 2.9 ± 3.1* |
| S. boulardii | 207 ± 17 | 196 ± 16 | 195 ± 24 | −5.7 ± 5.6 |
| Mesalazine | 207 ± 12 | 199 ± 18 | 201 ± 20 | −3.3 ± 5.5 |
| Infliximab | 208 ± 15 | 197 ± 14 | 197 ± 13 | −5.2 ± 3.4 |

*Significantly different from the positive control group (Dunnett, P < 0.05).

The greater impact of colitis on the animals in trial 1 probably left more room for improvement by any of the treatments. PB6 and mesalazine always resulted in a colon segment weight and a gross morphology score for the colon wall that could be clearly distincted from those of the colitis-positive control group (Table 12).

TABLE 12
Average gross morphology score for the colon wall and average
wet weight of a 5 cm colon segment.

| | Gross morphology score | Wet weight (g) |
|---|---|---|
| Trial 1 | | |
| IBD-negative control | 0.2 ± 0.4* | 0.378 ± 0.047* |
| IBD-positive control | 3.8 ± 0.8 | 1.406 ± 0.209 |
| PB6 1.5 × 10⁸ CFU/Kg | 1.0 ± 0.0* | 0.443 ± 0.063* |
| (post-induction) | | |
| PB6 1.5 × 10⁹ CFU/Kg | 0.6 ± 0.5* | 0.513 ± 0.317* |
| (post-induction) | | |
| Mesalazine | 0.8 ± 0.4* | 0.435 ± 0.052* |
| Infliximab | 3.0 ± 1.4 | 1.055 ± 0.490‘ |
| Trial 2 | | |
| IBD-negative control | 0.0 ± 0.0* | 0.274 ± 0.049* |
| IBD-positive control | 3.4 ± 1.1 | 0.832 ± 0.216 |
| PB6 1.5 × 10⁸ CFU/Kg | 0.6 ± 0.5* | 0.280 ± 0.039* |
| S. boulardii | 2.8 ± 1.3 | 0.735 ± 0.221 |
| Mesalazine | 1.2 ± 0.8* | 0.445 ± 0.145* |
| Infliximab | 2.0 ± 0.7 | 0.657 ± 0.076 |

*Significantly different from the positive control group (Dunnett, P < 0.05).

The health status of the colon wall in rats with TNBS induced colitis treated with PB6 and Mesacol was macroscopically the same as that in colitis-free rats. For an unknown reason mesalazine treatment was somewhat less effective in trial 2, resulting in some ulcerations. Visual examinations of the longitudinally opened colon segments clearly show the ulcerations and areas of necrotic tissue present in the positive control, the S. boulardii, and the infliximab treatment groups and the absence thereof in the PB6 groups and the trial 1 mesalazine group. In trial 1 one of the rats treated with infliximab had a gross morphology score of 1 and a colon segment weight of 0.402 g. These data suggest that in this particular animal the infliximab treatment was successful or that the induction of colitis was not. At the end of the treatment period the average body weight gain and colon segment weight data of rats treated with infliximab were intermediary to those of the colitis-negative and the colitis-positive control groups in both trials. This probably indicates that there was some effect of infliximab, although not statistically significant in these trials. In a similar trial in rats where infliximab was administered at the same dose of 3 mg/Kg i.v. 2 days before induction of colitis with TNBS, it was shown to have some effect on colon gross morphology but not to reduce the level of edema significantly below that of the colitis-positive control group.¹⁹ In the colon segments of rats treated with PB6 there were no ulcerations. Only hyperaemia was observed in the majority of these segments. PB6 clearly attenuates inflammation in the rat colon wall as induced by intrarectal administration of TNBS. The efficacy of a 7 days post-induction treatment with 3×1.5 10⁹ CFU/Kg/day observed in a previous trial was confirmed in trial 1.²⁰ While a 7 days post-induction treatment with PB6 3×8 10⁷ CFU/Kg/day from the same previous trial was concluded to be ineffective, an increase of the CFU/Kg/day to 3×1.5 10⁸ proved in both current trials to be sufficient to reduce inflammation to an extent that the data of this treatment could no longer be statistically discerned from those of the colitis-negative control group.

Conclusion

This study was designed and conducted to confirm and document the efficacy of Bacillus ‘PB6’ against 2,4,6-trinitrobenzenesulfonic acid (TNBS)-induced colitis in rats observed in a previous trial as well as to compare its efficacy with that of S. boulardii (probiotic), mesalazine and infliximab (standard drugs). This rat model is well established, reliable and widely used to examine the efficiency of drugs aimed at treating IBD. Without any treatment after induction of colitis several rats showed mild signs of diarrhea and on average kept losing weight throughout the remaining trial period. The gross morphology of the intestinal wall of their colon was characterized by inflammation, ulceration and even necrosis. Treatment with PB6 3 times 1.5 10⁸ CFU/Kg/day or 3 times 1.5 10⁹ CFU/Kg/day for 7 days resulted in a colon wall health status that was statistically identical to that of the negative control group and that of the group treated with the standard drug mesalazine, an anti-inflammatory.

### Example 4

### Antimicrobial Properties of the Metabolites of PB6

Methods and Materials

Antagonistic properties of Bacillus PB6 and Bacillus cereus isolated from Bactisubtil (Sanofi-synthelabo) were tested against different indicator strains e.g. C. perfringens ATCC13124, C. difficile ATCC9689 and Campylobacter jejuni ATCC 33291.

Bacillus cereus and Bacillus PB6 were each suspended in 5 ml of sterile saline. Using a swab a single streak of the probiotic suspensions on Tryptone Soy agar plates (Oxoid, Belgium) was made and the plates were incubated at 37° C. for 24 hours in aerobic conditions.

Afterwards a suspension of the different indicator strains were inoculated perpendicularly to both Bacillus cultures with a swab and incubated for 24 hours in aerobic conditions. Plates inoculated with Clostridia perfringens were incubated for 24 hours, plates inoculated with C. difficile, were incubated for 48 hours in anaerobic conditions using Anaerogen Pak (Oxoid, Belgium). A suspension of a 48 hours C. jejuni ATCC33291 culture was used to make 4 streaks perpendicularly to the probiotic cultures. Plates inoculated with Campylobacter species were incubated in micro-aerobic conditions (CampyGen, Oxoid) at 37° C. for 48 hours. The streak lines must not touch one another. Following incubation at 37° C., antagonistic effects were evaluated by the appearance of clear zones surrounding the junctions of the streak-lines indicating the inhibitory effect of one organism against the other.

Results and Discussion

PB6 had an antagonistic effect against C. perfringens ATCC13124 and C. difficile ATCC9689. A clear zone could be observed at the intersections of the streak-lines on the plate for both species. The antagonistic effect against C. perfringens is clearly visible due to the haemolytic characteristics of this species. An example is depicted in FIG. 1. Although not as clear on this picture, a significant clear zone at the intersection of PB6 and C. difficile cultures was also noticed.

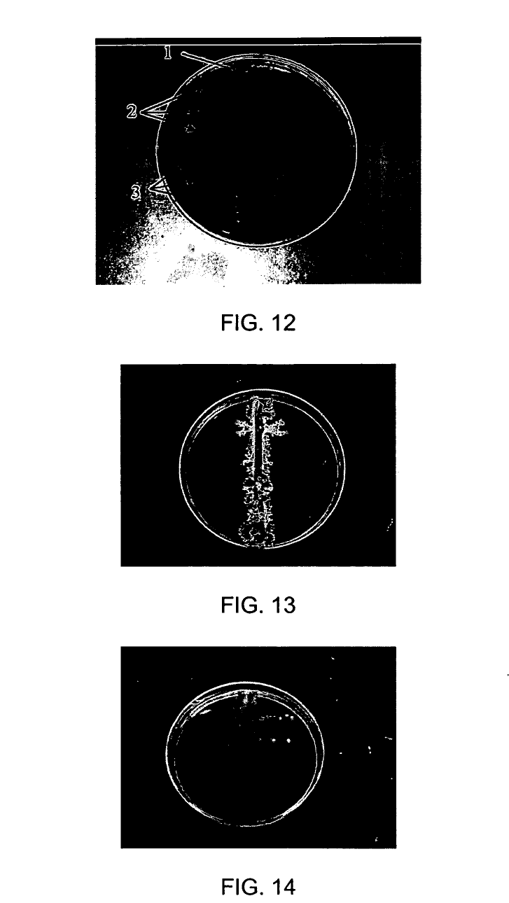

Bacillus cereus (Bactisubtil) had no antagonistic effect against C. perfringens ATCC13124 and C. difficile ATCC9689. No clear zone could be observed at the intersections of the streak-lines on the plate. An example of the test plate is depicted in FIG. 12.

Bacillus PB6 had an antagonistic effect against C. jejuni ATCC 33291. Clear zones can be observed at the intersections of the streak-lines on the plate. An example of the test plate is depicted in FIG. 13.

Bacillus cereus (Bactisubtil) had no antagonistic effect against C. jejuni as can be observed in FIG. 14.

Conclusion

Bacillus PB6 isolated from nature, clearly has strong antagonistic properties towards C. perfringens ATCC13124, C. difficile ATCC9689 and C. jejuni ATCC 33291. On the contrary, no effect could be observed for other human pathogenic bacteria tested. Bacillus cereus (Bactisubtil) did not show any antagonistic effect towards C. perfringens ATCC 13124, C. difficile ATCC9689 and other tested microorganisms.

Viable cells of the PB6 strain have been deposited with the American Type Culture Collection (“ATCC”), 10801 University Blvd., Manassas, Va., 20110-2209, U.S.A., on May 27, 2005, and assigned accession number PTA-6737. The deposit was made in accordance with 37 C.F.R. 1.801-1.809

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

### REFERENCES

¹McFarland L V. Epidemiology, risk factors and treatments for antibiotic-associated diarrhea. Dig Dis. 1998; 16:292-307.
²Bulusu M, Narayan S, Shetler K, Triadafilopoulos G. Leukocytosis as a harbinger and surrogate marker of Clostridium difficile infection in hospitalized patients with diarrhea. Am J Gastroenterol. 2000; 95:3137-3141.
³McFarland L V, Mulligan M E, Kwok R Y Y, Stamm W E. Nosocomial acquisition of Clostridium difficile infection. N Engl J Med. 1989; 320:204-210.
⁴Cheng S H, Lu J J, Young T G, Perng C L, Chi W M. Clostridium difficile-associated diseases: comparison of symptomatic infection versus carriage on the basis of risk factors, toxin production, and genotyping results. Clin Infect Dis. 1997; 25:157-158.
⁵Hall, I. C., O'Tool, E. Intestinal flora in newborn infants with a description of a new pathogenic anaerobe, Bacillus difficilis. AJDC. 1935; 49:390-402
⁶Lyerly, D. M.; Krivan, H. C., Wilkins, T. D. Clostridium difficile its disease and toxins. Clin Microbiol Rev 1988; 1: 1-18
⁷Gerding, D. N. Disease associated with Clostridium difficile infection Ann Intern Med 1989; 110:255-257
⁸McFarland, L. V. Stamm, W. E. Review of Clostridium difficile-associated diseases. AM J Infect Control 1986; 14:99-109
⁹Anand, A., Glatt A. E. Clostridium difficile infection associated with antineoplastic chemotherapy: a review. Clin Infect Dis. 1993; 17:109-113
¹⁰Sharma, A. K., Holer, F. E. Clostridium difficile diarrhea after use of tacrolimus following renal transplantation. Clin Infect Dis. 1998; 27:1540-1541.
¹¹Kelly, C. P. LaMont, J. T. Clostridium difficile infection. Annu Rev 1998; 49:375-390
¹²Kelly, C. P., Pothoulakis, K., LaMont, J. T. Clostridium Dijficile colitis. N Engl J Med. 1994; 330:257-262
¹³Anand, A., Bashey, B., Mir, T., Glatt, A. E. Epidemiology, clinical manifestations, and outcome of Clostridium difficile-associated diarrhea. Am J Gastroenterol. 1994; 89:519-523
¹⁴Kyne, L. et al. Asymptomatic carriage of Clostridium difficile and serum levels of IgG antibody against toxin A. N Engl J Med. 2000; 342:390-397
¹⁵Bartlett, J. G. Antibiotic-associated diarrhea. Clin Infect Dis. 1992.15/573-581
¹⁶Rubin, M. S. et al Severe Clostridium difficile colitis. Dis Colon Rectum. 1995; 38:350-354
¹⁷Anand, A. et al Epidemiology, clinical manifestations, and outcome of Clostridium difficile-associated diarrhea. Am J Gastroenterol. 1994; 89:519-523
¹⁸Wilcox, M. H. et al Financial burden of hospital-acquired Clostridium difficile infections. J Hosp Infect. 1996; 34:23-30
¹⁹Riley, T. V. et al Community-acquired Clostridium difficile-associated diarrhea. Clin Infect Dis. 1995; 20 (suppl 2):S263-S265
²⁰Samore, M. H. et al Clinical and molecular epidemiology of sporadic and clustered cases of nosocomial Clostridium difficile diarrhea. Am J Med. 1996; 100:32-40
²¹Djuretic, T. et al Infectious intestinal disease in elderly people. Commun Dis Rep CDR Wkly. 1996; 19:R107-R112
²²Olson, M. M. et al Ten years of prospective Clostridium difficile-associated disease surveillance and treatment at the Minneapolis A Medical Cener, 1982-1991. Infect Control Hosp Epidemiol 1994; 15(6):371-381
²³Teasley, D. G. et al. Prospective randomized trial of metronidazole versus vancomycin for Clostridium difficile-associated diarrhea and colitis. Lancet 1983; 2(8358):1043-1046
²⁴Gerding, D. N. Is there a relationship between vancomycin-resistant enterococcal infection and Clostridium difficile infection? Clin Infect Dis 1997; 25 Suppl 2:S206-210
²⁵Kelly, C. P. et al. Clostridium difficile colitis. N Engl J Med 1994; 330(4):257-262
²⁶Fekety, R. and Shah, A. B. Diagnosis and treatment of Clostridium difficile colitis. JAMA 1993; 269(1):71-75
²⁷Pothoulakis, C. and LaMont, J. T. Clostridium difficile colitis and diarrhea. Gastroenterol Clin North Am 1993; 22(3):623-637
²⁸Fekety, R. et al. Recurrent Clostridium difficile diarrhea: characteristics of and risk factors for patients enrolled in a prospective, randomized, double-blinded trial. Clin Infect Dis 1997; 24(3):324-333
²⁹Carmeli, Y. et al. Antecedent treatment with different antibiotic agents as a risk factor for vancmycin resistant enterococcus. Emerg Infect Dis 2002; 8:802-807
³⁰Gerding, D. N. Is there a relationship between vancomycin-resistant enterococcal infection and Clostridium difficile infection? Clin Infect Dis 1997; 25 Suppl 2:S206-210
³¹Poduval, R. D. et al. Clostridium difficile and vancomycin-resistant enteroccoccus: the new nosocomial alliance. Am J Gastroenterol 2000; 95:3513-3515
³²Pelaez, T. et al. Metronidazole Resistance in Clostridium difficile: a new emerging problem? 38th Interscience Conference on antimicrobial agents and chemotherapy. Sept. 24-27, San Diego, Calif., American Society for Microbiology
³³Pinchuk, I. et al. In Vitro Anti-Helicobacter pylori Activity of the Probiotic Strain Bacillus subtilis 3 Is Due to Secretion of Antibiotics. Antimicrob. Agents Chemother. 2001; 45: 3156-3161
³⁴Caruso A., Flamminio S., Folghera S., Peroni L., Foresti I., Balsari A., Turano A. Expression of activation markets on peripheral-blood lymphocytes following oral administration of Bacillus subtilis spores. Intl. J. Immunopharmacol. (1993) 15, 87-92
³⁵Ciprandi G., Scordamaglia A., Venuti D., Caria M., Caninica G. W. In vitro effects of Bacillus subtilis on the immune response. Chemioterapia (1986) 5, 404-407.
³⁶Grasso G., Migliaccio P., Tanganelli C., Brugo M. A., Muscettola M. Restorative effect of Bacillus subtilis spores on interferon production in aged mice. Ann. N.Y. Acad. Sci. (1994) 717, 198-208
³⁷Duc L. H., Hong H. A., Cutting S. M. Germination of the spores in the gastroinstestinal tract provides a novel route for heterologous antigen delivery. Vaccine (2003) 21 (27-30), 4215-4224
³⁸Duc L. H., Hong H. A., Uyen N. Q., Cutting S. M. Intracellular fate and immunogenicity of Bacillus subtilis spores. Vaccine (2004), 22(15-16), 1873-1885.
³⁹Fais S., Pallone F., Nava C., Magnani M. Lymphocyte activation by B. subtilis spores. Boll. Ist. Sieroter. Milan (1987) 66(5), 391-394
⁴⁰Prokesova L., Novakova M., Julak J., Mara M. Effect of Bacillus firmus and other sporulating aerobic microorganisms on in vitro stimulation of human lymphocytes: a comparative study. Folia Microbiol (1994) 39, 501-504
⁴¹Kosaka T., Maeda T., Nakada Y., Yukawa M., Tanaka S. Effect of Bacillus subtilis spore administration on activation of macrophages and natural killer cells in mice. Vet. Microbiol. (1998) 60(2-4), 215-225.
⁴²Paul A. A. C. Inulin and Oligofructose: Safe Intakes and Legal Status. Journal of Nutrition (1999) 129, 1412S-1417S.
⁴³Gibson G. R., Probert H. M., Loo J. V., Rastall R. A., Roberfroid M. B. Dietary modulation of the human colonic microbacteria: updating the concept of probiotics. Nutr. Res. Rev. (2004) 17, 259-275
⁴⁴Gibson G. R., Roberfroid M. B. Dietary modulation of the human colonic microbiota—introducing the concept of probiotic. J. Nutr. (1995) 125, 1401-1412
⁴⁵Roberfroid M. Dietary Fiber, Inulin, and Oligofructose: a Review Comparing their Physiological Effects. Crit. Rev. Food Sci. Nutri. (1993) 33, 103-148
⁴⁶Van Loo J., Coussement P., De Leenheer L., Hoebregs H., Smits G. On the presence of inulin and oligofructose as natural ingredients in the Western diet. Crit. Rev. Food Sci. Nutr. (1995) 35, 525-552
⁴⁷Ezaki, T., Hashimoto, Y. and Yabuuchi, E. Fluorometric deoxyribonucleic acid-deoxyribonucleic acid hybridization in microdilution wells as an alternative to membrane filter hybridization in which radioisotopes are used to determine genetic relatedness among bacterial strains. Int. J. Syst. Bacteriol. 1989; 39: 224-229
⁴⁸Goris, J., Suzuki, K., De Vos, P., Nakase, T. and Kersters, K. Evaluation of a microplate DNA-DNA hybridization method compared with the initial renaturation method. Can J. Microbiol. 1998; 44: 1148-1153
⁴⁹Wayne et al. Int. J. Syst. Bacteriol. 1987; 37: 463-464
⁵⁰Smith L. D. S., Williams B. L. 1984. The Pathogenic Anaerobic Bacteria, 3^{rd} ed. Charles C. Thomas, Springfield, Ill.
⁵¹Gorbach S. L. 1998. Gas gangrene and other clostridial skin and soft tissue infections, p. 915-922. In Gorbach S. L, Bartlett J. G., and Blacklow N. R. (ed.), Infectious Diseases, 2^{nd} ed. W.B. Saunders Company, Philadelphia, Pa.
⁵²Anand A., Glatt A. E. 1993. Clostridium difficile infection associated with antineoplastic chemotherapy: a review. Clin. Infect. Dis. 17:109-113.
⁵³Sharma A. K., Holder F. E. 1998. Clostridium difficile diarrhea after use of tacrolimus following renal transplantation. Clin. Infect. Dis. 27: 1540-1541.
⁵⁴Johnson S., Gerding D. N. 1998. Clostridium difficile-associated diarrhea. Clin. Infect. Dis. 26:1027-1034.
⁵⁵Bartlett J. G. 2002. Clinical practice Antibiotic-associated diarrhea. N. Eng. J. Med. 346:334-339.
⁵⁶Dzink J., Bartlett J. G. 1980. In vitro susceptibility of C. difficile isolates from patients with antibiotic-associated diarrhea or colitis. Antimicrob. Agents Chemother. 17:695-698.
⁵⁷Tannock G. W. 1999. Probiotics: A critical review. Horizon Scientific Press.
⁵⁸Kim, K. et al. Suppression of inflammatory responses by surfactin, a selective inhibitor of platelet cytosolic phospholipase A₂. Biochem. Pharmacol. 1998; 55: 975-985.

We claim:

1. A method for the prophylaxis of a bowel condition, comprising the step of administering as a probiotic an effective amount of a Bacillus bacteria that produces lipopeptides.

2. A method as defined in claim 1, wherein the bowel condition is selected from the group consisting of antibiotic associated diarrhea, Clostridium difficile acquired diarrhea, inflammatory bowel disease, and gastro-intestinal disease.

3. A method for the treatment of a bowel condition, comprising the step of administering as a probiotic an effective amount of a Bacillus bacteria that produces lipopeptides.

4. A method as defined in claim 3, wherein the bowel condition is selected from the group consisting of antibiotic associated diarrhea, Clostridium difficile acquired diarrhea, inflammatory bowel disease, and gastro-intestinal disease.

5. The method as defined in claim 1, further comprising the administration of inulin combined with the Bacillus bacteria.

6. The method as defined in claim 3, further comprising the administration of inulin combined with the Bacillus bacteria.

7. The method as defined in claim 1, further comprising the administration of a probiotic combined with the Bacillus bacteria.

8. The method as defined in claim 3, further comprising the administration of a probiotic combined with the Bacillus bacteria.

9. The method as defined in claim 1, wherein the bacteria are selected from the group consisting of Bacillus amyloliquefaciens and Bacillus subtilis.

10. The method as defined in claim 3, wherein the bacteria are selected from the group consisting of Bacillus amyloliquefaciens and Bacillus subtilis.

11. The method as defined in claim 1, wherein the Bacillus bacteria produce a synergistic compound upon administration.

12. The method as defined in claim 3, wherein the Bacillus bacteria produce a synergistic compound upon administration.

13. An isolated bacterial strain having the 16S rRNA sequence of SEQ ID NO. 1.

14. An isolated bacterial strain as defined in claim 13, wherein the strain has at least 90% homology to the 16S rRNA sequence of SEQ ID NO. 1.

15. An isolated bacterial strain having the partial gyrA sequence of SEQ ID NO. 2.

16. An isolated bacterial strain as defined in claim 15, wherein the strain has at least 90% homology to the partial gyrA sequence of SEQ ID NO. 2.

17. An isolated bacterial strain having the partial gyrA sequence of SEQ ID NO. 3.

18. An isolated bacterial strain as defined in claim 17, wherein the strain has at least 90% homology to the partial gyrA sequence of SEQ ID NO. 3.

19. A method as defined in claim 1, wherein the Bacillus bacteria consist of bacteria of the strain identified as ATCC strain PTA-6737.

20. A method as defined in claim 3, wherein the Bacillus bacteria consist of bacteria of the strain identified as ATCC strain PTA-6737.


SEQUENCE LISTING (3 sequences)

SEQ ID NO: 1 - DNA, 1448 bp, Bacillus subtilis
aaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgtgacctcaccaactagctaatgcgccgcgggtccatctgtaagtggtagccgaagccaccttttatgtctgaaccatgcggttcaaacaaccatccggtattagccccggtttcccggagttatcccagtcttacaggcaggttacccacgtgttactcacccgtccgccgctaacatcagggagcaagctcccatctgtccgctcgacttgcatgtattaggcacgccgccagcgttcgtcctgagccatggatcaa

SEQ ID NO: 2 - DNA, 1016 bp, Bacillus subtilis
gtcaggaaatgcgtacgtccttcctggactatgcaatgagcgttatcgtatcccgggcgcttccggatgtgcgtgacggtctgaagccggttcacagacggattttgtacgcaatgaatgatttaggcatgaccagtgacaaaccatataaaaaatctgcccgtatcgtcggtgaagttatcggtaagtaccacccgcacggtgactcagcggtttacgaatcaatggtcagaatggcgcaggattttaactaccgctacatgcttgttgacggacacggcaacttcggttcggttgacggcgactcagcggccgcgatgcgttacacagaagcgagaatgtcaaaaatcgcaatggaaattctgcgtgacattacgaaagacacgattgactatcaagataactatgacggttcagaaagagagcctgccgtcatgccttcgagatttccgaatctgctcgtaaacggggctgccggtattgcggtcggaatggcgacaaacattcccccgcatcagcttggagaagtcattgaaggcgtgcttgccgtaagtgagaatcctgagattacaaaccaggagctgatggagtacatcccgggcccggattttccgactgcaggtcagattttgggccggagcggcatccgcaaggcgtatgaatccggacggggatcaatcacaatccgggctaaggctgaaatcgaagagacttcatcgggaaaagaaagaattattgtcacggaacttccttatcaggtgaacaaagcgagattaattgaaaaaatcgcggatcttgtccgggacaaaaaaatcgaaggaattaccgatctgcgagacgaatccgaccgtaacggaatgagaatcgtcattgagatccgccgtgacgccaatgctcacgtcattttgaataacctgtacaaacaaacggccctgcagacgtctttcgggatcaacctgctggcgctcgttgacggacagccgaaggtactaagcctgaagcaatgcctggagcat

SEQ ID NO: 3 - DNA, 801 bp, Bacillus subtilis
ataaaaaatctgcccgtatcgtcggtgaagttatcggtaagtaccacccgcacggtgactcagcggtttacgaatcaatggtcagaatggcgcaggattttaactaccgctacatgcttgttgacggacacggcaacttcggttcggttgacggcgactcagcggccgcgatgcgttacacagaagcgagaatgtcaaaaatcgcaatggaaattctgcgtgacattacgaaagacacgattgactatcaagataactatgacggttcagaaagagagcctgccgtcatgccttcgagatttccgaatctgctcgtaaacggggctgccggtattgcggtcggaatggcgacaaacattcccccgcatcagcttggagaagtcattgaaggcgtgcttgccgtaagtgagaatcctgagattacaaaccaggagctgatggagtacatcccgggcccggattttccgactgcaggtcagattttgggccggagcggcatccgcaaggcgtatgaatccggacggggatcaatcacaatccgggctaaggctgaaatcgaagagacttcatcgggaaaagaaagaattattgtcacggaacttccttatcaggtgaacaaagcgagattaattgaaaaaatcgcggatcttgtccgggacaaaaaaatcgaaggaattaccgatctgcgagacgaatccgaccgtaacggaatgagaatcgtcattgagatccgccgkgacgccaatgctcacgtcattttgaataacctgtacaamcaaacggccctgcagaystctttcg